SEQUENCE LISTING (77 sequences)

SEQ ID NO: 1 - protein, 8 aa, synthetic construct
GFNIKDTY

SEQ ID NO: 2 - protein, 8 aa, synthetic construct
IEPMNGNT

SEQ ID NO: 3 - protein, 13 aa, synthetic construct
SRGGWLLLYVMDY

SEQ ID NO: 4 - protein, 11 aa, synthetic construct
KSLLHSNGNTY

SEQ ID NO: 5 - protein, 9 aa, synthetic construct
FQQLEYPYT

SEQ ID NO: 6 - protein, 120 aa, synthetic construct
QVQLVQSGAEVVKPGASVKISCKASGFNIKDTYIYWVRQRTGQGLEWIGRIEPMNGNTKYDPKFQGRLTMTANTSKNTAYLQLSSLTSEDTAVYYCSRGGWLLLYVMDYWGQGTTVTVSS

SEQ ID NO: 7 - protein, 112 aa, synthetic construct
DIVMTQSASSLPVTPGEPASISCRSDKSLLHSNGNTYLYWFLQKPGQSPQVLIYRMSNLASGVPDRFSGSGSGTDFTLTISRVEAEDVGVYYCFQQLEYPYTFGQGTKLEIK

SEQ ID NO: 8 - protein, 120 aa, synthetic construct
EVQLVQSGAELVKPGASVKLSCAASGFNIKDTYIYWVRQAPGQGLEWVGRIEPMNGNTKYDPKFQGRFTMTANTSKNTAYLQLSSLRSEDTAVYYCSRGGWLLLYVMDYWGQGTLVTVSS

SEQ ID NO: 9 - protein, 112 aa, synthetic construct
DIVMTQSPSSLSVTPGERVSISCRSDKSLLHSNGNTYLYWFLQKPGQSPQVLIYRMSNLASGVPDRFSGSGSGTDFTLTISRVEAEDVGVYYCFQQLEYPYTFGQGTKLEIK

SEQ ID NO: 10 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 11 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 12 - protein, 15 aa, synthetic construct
GGGGSGGGGSGGGGS

SEQ ID NO: 13 - protein, 5 aa, synthetic construct
GGGGS

SEQ ID NO: 14 - protein, 492 aa, synthetic construct
DIVMTQSASSLPVTPGEPASISCRSDKSLLHSNGNTYLYWFLQKPGQSPQVLIYRMSNLASGVPDRFSGSGSGTDFTLTISRVEAEDVGVYYCFQQLEYPYTFGQGTKLEIKGGGGSGGGGSGGGGSQVQLVQSGAEVVKPGASVKISCKASGFNIKDTYIYWVRQRTGQGLEWIGRIEPMNGNTKYDPKFQGRLTMTANTSKNTAYLQLSSLTSEDTAVYYCSRGGWLLLYVMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 15 - protein, 492 aa, synthetic construct
DIVMTQSPSSLSVTPGERVSISCRSDKSLLHSNGNTYLYWFLQKPGQSPQVLIYRMSNLASGVPDRFSGSGSGTDFTLTISRVEAEDVGVYYCFQQLEYPYTFGQGTKLEIKGGGGSGGGGSGGGGSEVQLVQSGAELVKPGASVKLSCAASGFNIKDTYIYWVRQAPGQGLEWVGRIEPMNGNTKYDPKFQGRFTMTANTSKNTAYLQLSSLRSEDTAVYYCSRGGWLLLYVMDYWGQGTLVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 16 - protein, 246 aa, synthetic construct
DIVLTQSPASLAVSLGQRATISCRASESVEYYGTSLMQWYQQKPGQPPKLLIYTASNVESGVPVRFSGSGSGTDFSLNIHPVEEDDIAMYFCQQSRKVPYTFGGGTKLEIKGGGGSGGGGSGGGGSQVQLQQSGPELMKPGASVKISCKATGYSFSGFWIEWVKQRPGHGLEWIGEILPGSGASNYHENFKGRATITADTSANTAYMQLRSLTSEDSAVYYCARERYYGSKYFDVWGAGTTVTVSS

SEQ ID NO: 17 - protein, 251 aa, synthetic construct
DIVMTQAAPSVPVTPGESVSISCRSSKSLLHSNGNTYLYWFLQRPGQSPQLLISQMSNLASGIPDRFSGSGSGTAFTLRISRVEAEDVGVYYCMQHLEYPFTFGSGTKLEIKGGGGSGGGGSGGGGSEVQLQQSGAELVKPGASVKLSCTASGFNIKDNYMNWVKQRPEQGLEWIGRIDPANGNPKYDPKFQGKATITADTSSNTAYLQLSSLTSEDTAVYYCTRWIYYYGSRYEWYFDVWGAGTTVTVSS

SEQ ID NO: 18 - protein, 247 aa, synthetic construct
DIVMTQAAPSVSVTPGESVSISCRSDKSLLHSNGNTYLYWFLQRPGQSPQVLIYRMSNLASGVPDRFSGSGSGTGFTLRVSRVEAEDVGVYYCFQQLEYPYTFGGGTKLEIKGGGGSGGGGSGGGGSEVQLQQSGAELVKPGASVKLSCTASGFNIKDTYIYWVKQRPGQGLEWIGRIEPMNGNTKYDPKFQGKATMTANTSSNTAYLHLSSLASEDTAVYFCSRGGWLLLYVMDYWGQGTSVTVSS

SEQ ID NO: 19 - protein, 242 aa, synthetic construct
DIQMTQSPASLSASVGESVTITCRASENIYSYLTWYQQKQGKSPQLLVYNAKTLTEDVPSRFSGSGSGTQFSLKINSLQPEDFGSYYCQHHYGSPRTFGGGTKLEIKGGGGSGGGGSGGGGSQAQLQQSGAELMKPGASVKISCKATGYTFSNFWIEWVVQRPGHGLEWIGEILPGRGTTNYNEKFRGKATFTADTSSDTAYIQLSSLTSEDSAVYYCARERFYGSKYFDVWGAGTTVTVSS

SEQ ID NO: 20 - protein, 491 aa, synthetic construct
DIVLTQSPASLAVSLGQRATISCRASESVEYYGTSLMQWYQQKPGQPPKLLIYTASNVESGVPVRFSGSGSGTDFSLNIHPVEEDDIAMYFCQQSRKVPYTFGGGTKLEIKGGGGSGGGGSGGGGSQVQLQQSGPELMKPGASVKISCKATGYSFSGFWIEWVKQRPGHGLEWIGEILPGSGASNYHENFKGRATITADTSANTAYMQLRSLTSEDSAVYYCARERYYGSKYFDVWGAGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 21 - protein, 496 aa, synthetic construct
DIVMTQAAPSVPVTPGESVSISCRSSKSLLHSNGNTYLYWFLQRPGQSPQLLISQMSNLASGIPDRFSGSGSGTAFTLRISRVEAEDVGVYYCMQHLEYPFTFGSGTKLEIKGGGGSGGGGSGGGGSEVQLQQSGAELVKPGASVKLSCTASGFNIKDNYMNWVKQRPEQGLEWIGRIDPANGNPKYDPKFQGKATITADTSSNTAYLQLSSLTSEDTAVYYCTRWIYYYGSRYEWYFDVWGAGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 22 - protein, 492 aa, synthetic construct
DIVMTQAAPSVSVTPGESVSISCRSDKSLLHSNGNTYLYWFLQRPGQSPQVLIYRMSNLASGVPDRFSGSGSGTGFTLRVSRVEAEDVGVYYCFQQLEYPYTFGGGTKLEIKGGGGSGGGGSGGGGSEVQLQQSGAELVKPGASVKLSCTASGFNIKDTYIYWVKQRPGQGLEWIGRIEPMNGNTKYDPKFQGKATMTANTSSNTAYLHLSSLASEDTAVYFCSRGGWLLLYVMDYWGQGTSVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 23 - protein, 487 aa, synthetic construct
DIQMTQSPASLSASVGESVTITCRASENIYSYLTWYQQKQGKSPQLLVYNAKTLTEDVPSRFSGSGSGTQFSLKINSLQPEDFGSYYCQHHYGSPRTFGGGTKLEIKGGGGSGGGGSGGGGSQAQLQQSGAELMKPGASVKISCKATGYTFSNFWIEWVVQRPGHGLEWIGEILPGRGTTNYNEKFRGKATFTADTSSDTAYIQLSSLTSEDSAVYYCARERFYGSKYFDVWGAGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 24 - protein, 20 aa, synthetic construct
METDTLLLWVLLLWVPGSTG

SEQ ID NO: 25 - DNA, 798 bp, synthetic construct
  source 1..798: mol_type = other DNA
atggagaccgacacactgctgctgtgggtgctgctgctgtgggtgccaggatccaccggcgacatcgtgctgacacagtcccctgctagcctggctgtgagcctgggccagagagccacaatctcttgccgcgctagcgagtctgtggagtactatggcaccagcctgatgcagtggtatcagcagaagcctggccagccccctaagctgctgatctacacagcctctaacgtggagtccggcgtgccagtgagattctccggcagcggctctggcaccgacttttccctgaatatccaccccgtggaggaggacgacatcgccatgtacttctgccagcagagccgcaaggtgccttatacctttggcggcggcacaaagctggagatcaagggcggcggcggctctggaggaggaggatccggaggaggaggaagccaggtgcagctgcagcagtctggaccagagctgatgaagccaggagctagcgtgaagatctcttgtaaggctaccggctattccttcagcggcttttggatcgagtgggtgaagcagaggccaggacacggactggagtggatcggagagatcctgccaggatctggagcttccaactatcatgagaatttcaagggcagggccaccatcacagctgacaccagcgccaacacagcttacatgcagctgcggagcctgacatctgaggattccgccgtgtactattgcgctagggagcggtactatggctctaagtactttgacgtgtggggagctggaaccacagtgaccgtgtccagc

SEQ ID NO: 26 - DNA, 813 bp, synthetic construct
  source 1..813: mol_type = other DNA
atggagaccgacacactgctgctgtgggtgctgctgctgtgggtgcctggctctaccggcgacatcgtgatgacacaggctgctccatccgtgcctgtgacaccaggcgagagcgtgtctatctcctgcagatccagcaagtctctgctgcactccaacggcaatacctatctgtactggttcctgcagaggccaggacagagccctcagctgctgatctctcagatgtccaacctggcctctggcatccccgaccggttcagcggatctggatccggcaccgcctttacactgaggatcagccgggtggaggctgaggatgtgggcgtgtactattgcatgcagcatctggagtaccctttcacctttggctctggcacaaagctggagatcaagggaggaggaggatccggaggaggaggaagcggtggcggcggctctgaggtgcagctgcagcagtccggagctgagctggtgaagccaggagctagcgtgaagctgtcttgtaccgcctccggcttcaacatcaaggacaactacatgaattgggtgaagcagagaccagagcagggactggagtggatcggaaggatcgaccctgctaacggcaatccaaagtacgatcccaagtttcagggcaaggccaccatcacagctgacacctcttccaatacagcctatctgcagctgagctctctgacctccgaggatacagccgtgtactattgcacaagatggatctactattacggcagccgctatgagtggtactttgacgtgtggggagctggaaccacagtgaccgtgtccagc

SEQ ID NO: 27 - DNA, 801 bp, synthetic construct
  source 1..801: mol_type = other DNA
atggagaccgacacactgctgctgtgggtgctgctgctgtgggtgccaggaagcaccggcgacatcgtgatgacacaggctgctccatccgtgtccgtgacaccaggcgagagcgtgtctatctcctgccgctctgataagtccctgctgcacagcaacggcaatacctacctgtattggttcctgcagagacccggccagtctcctcaggtgctgatctaccgcatgtctaacctggcctccggcgtgccagataggttcagcggatctggatccggaaccggctttacactgagggtgtctcgggtggaggctgaggacgtgggcgtgtactattgcttccagcagctggagtacccctatacctttggcggcggcacaaagctggagatcaagggaggaggaggatccggaggaggaggaagcggtggcggcggctctgaggtgcagctgcagcagtccggagctgagctggtgaagccaggagctagcgtgaagctgtcttgtaccgcctccggctttaatatcaaggatacatacatctattgggtgaagcagaggcctggacagggactggagtggatcggaaggatcgagcccatgaacggcaataccaagtacgaccctaagttccagggcaaggccaccatgacagctaacacctccagcaatacagcttatctgcatctgtcttccctggcctctgaggacaccgccgtgtacttttgctcccgcggaggatggctgctgctgtacgtgatggattattggggccagggcaccagcgtgacagtgagctct

SEQ ID NO: 28 - DNA, 786 bp, synthetic construct
  source 1..786: mol_type = other DNA
atggagaccgacacactgctgctgtgggtgctgctgctgtgggtgccaggaagcaccggcgacatccagatgacacagtctccagcttctctgtccgctagcgtgggagagtccgtgaccatcacatgcagagctagcgagaacatctactcttatctgacctggtatcagcagaagcagggcaagtctccacagctgctggtgtacaatgccaagaccctgacagaggatgtgccatccaggttctctggatccggaagcggaacccagttttccctgaagatcaacagcctgcagcctgaggacttcggctcctactattgccagcaccattacggaagcccacggacctttggcggaggaacaaagctggagatcaagggcggcggcggctctggaggaggaggatccggaggaggaggaagccaggcccagctgcagcagagcggcgctgagctgatgaagccaggcgcctccgtgaagatcagctgtaaggctaccggctacacattctctaatttttggatcgagtgggtggtgcagaggcctggacacggactggagtggatcggagagatcctgccaggaaggggaaccacaaactataatgagaagttccggggcaaggccacctttacagctgacacctccagcgatacagcctacatccagctgtcttccctgacatccgaggacagcgccgtgtactattgcgctagggagcggttctacggctctaagtattttgacgtgtggggcgctggcaccacagtgaccgtgagctct

SEQ ID NO: 29 - DNA, 720 bp, synthetic construct
  source 1..720: mol_type = other DNA
gatgtgcagctggtgcagagcggagctgaggtgaagaagccaggagcttctgtgaaggtgtcctgcaaagccagcggctacaccttcacaagatataccatgcactgggtgcgccaggctcctggacagggcctggaatggatcggctatatcaatccaagcagaggctacaccaactatgccgactctgtgaagggccgctttacaatcaccaccgataagtccaccagcacagcttacatggagctgagctctctgaggtccgaggacaccgccacatactattgcgctcggtactatgacgatcattactgtctggattattggggtcagggtaccacagtgaccgtgtcctctggcggcggcggcagcggcggaggaggctccggtggcggcggctccgacatcgtgctgacccagtcccctgccacactgtctctgtccccaggagagagagccaccctgagctgcagggctagccagtccgtgtcctacatgaattggtaccagcagaagccaggcaaggctcccaagaggtggatctatgatacatctaaggtggcctccggagtgcctgctcggttcagcggatctggatccggaaccgactacagcctgacaatcaattctctggaggccgaggatgccgctacctactattgtcagcagtggtcttccaacccactgacctttggcggcggcacaaaggtggagatcaag

SEQ ID NO: 30 - DNA, 15 bp, synthetic construct
  source 1..15: mol_type = other DNA
ggaggaggaggatcc

SEQ ID NO: 31 - protein, 120 aa, synthetic construct
EVQLQQSGAELVKPGASVKLSCTASGFNIKDTYIYWVKQRPGQGLEWIGRIEPMNGNTKYDPKFQGKATMTANTSSNTAYLHLSSLASEDTAVYFCSRGGWLLLYVMDYWGQGTSVTVSS

SEQ ID NO: 32 - protein, 113 aa, synthetic construct
DIVMTQAAPSVSVTPGESVSISCRSDKSLLHSNGNTYLYWFLQRPGQSPQVLIYRMSNLASGVPDRFSGSGSGTGFTLRVSRVEAEDVGVYYCFQQLEYPYTFGGGTKLEIKR

SEQ ID NO: 33 - protein, 117 aa, synthetic construct
EVQLQEAGPSLVKPSQTLSLTCFVTGDSITSGYWHWIRKFPGNKLEYMGYINYSGYTYYNPSLKSRFSITRDTSKNQYYLQLNSVTTEDTATYYCTSERNWGAFAYWGQGTLITVSS

SEQ ID NO: 34 - protein, 108 aa, synthetic construct
ENVLTQSPAIMSASPGEKVTMTCRASSSVISTYLHWYQQKSGASPKLWIYSTSNLASGVPARFSGSGSGTSYSLTISSVEAEDAATYYCQQFSGYPYTFGGGTKLEIK

SEQ ID NO: 35 - protein, 112 aa, synthetic construct
DIVMTQSASSLPVTPGESVSISCRSDKSLLHSNGNTYLYWFLQRPGQSPQVLIYRMSNLASGVPDRFSGSGSGTEFTLTISRVEAEDVGVYYCFQQLEYPYTFGPGTKLEIK

SEQ ID NO: 36 - protein, 120 aa, synthetic construct
QVQLLQSGAEVVKPGASVKISCKASGFNIKDTYIYWVKQPTGQGLEWIGRIEPMNGNTKYDPKFQGRFTMTANTSINTAYLQLSSLTSEDTAVYYCSRGGWLLLYVMDYWGQGTTVTVSS

SEQ ID NO: 37 - protein, 112 aa, synthetic construct
DIVMTQSPLSLSVTPGEPASISCRSDKSLLHSNGNTYLYWFLQKPGQSPQVLIYRMSNLASGVPDRFSGSGSGTDFTLKISRVEAEDVGVYYCFQQLEYPYTFGQGTKLEIK

SEQ ID NO: 38 - protein, 120 aa, synthetic construct
QVQLVQSGAELVKPGASVKLSCKASGFNIKDTYIYWVRQAPGQGLEWIGRIEPMNGNTKYDPKFQGRVTMTANTSINTAYLELSSLRSEDTAVYYCSRGGWLLLYVMDYWGQGTLVTVSS

SEQ ID NO: 39 - protein, 108 aa, synthetic construct
ENVLTQSPATLSVSPGEPVTLNCRASSSVISTYLHWYQQKPGQAPRLWIYSTSNLASGIPARFSGSGSGTEYTLTISSVESEDVAVYYCQQFSGYPYTFGQGTKLEIK

SEQ ID NO: 40 - protein, 117 aa, synthetic construct
QVQLQESGPELVKPSQTLSLTCKVSGDSITSGYWHWIRQFPGRKLEWVGYINYSGYTYYNPSLKSRLTISRDTSKNQYFLELRSVTSDDTAVYYCTSERNWGAFAYWGQGTLITVSS

SEQ ID NO: 41 - protein, 108 aa, synthetic construct
ENVLTQSPASLSVSPGERVTLNCRASSSVISTYLHWYQQKPGKPPRLWIYSTSNLASGIPARFSGSGSGTEYTLTISSVESEDFAVYYCQQFSGYPYTFGQGTKLEIK

SEQ ID NO: 42 - protein, 117 aa, synthetic construct
QVQLQESGPELVKPSETLSLTCKVSGDSITSGYWHWIRQFPGQKLEYMGYINYSGYTYYNPSLKSRLTISRDTSKNQYFLKLRSVTSDDTALYYCTSERNWGAFAYWGQGTLITVSS

SEQ ID NO: 43 - protein, 108 aa, synthetic construct
ENVLTQSPATLSASPGERATLSCRASSSVISTYLHWYQQKPGQAPKLWIYSTSNLASGVPARFSGSGSGTDYTLTISSVEPEDFAVYYCQQFSGYPYTFGQGTKLEIK

SEQ ID NO: 44 - protein, 117 aa, synthetic construct
QVQLQESGPGLVKPSETLSLTCTVSGDSITSGYWHWIRQPPGKGLEWIGYINYSGYTYYNPSLKSRFTISRDTSKNQYYLQLNSVTAEDTAVYYCTSERNWGAFAYWGQGTLVTVSS

SEQ ID NO: 45 - protein, 108 aa, synthetic construct
ENVLTQSPASLSASPGERVTITCRASSSVISTYLHWYQQKPGQAPKLWIYSTSNLASGVPARFSGSGSGTDYTLTISSVEPEDFATYYCQQFSGYPYTFGQGTKLEIK

SEQ ID NO: 46 - protein, 117 aa, synthetic construct
EVQLQESGPGLVKPSGTLSLTCAVSGDSITSGYWHWIRQAPGKGLEWVGYINYSGYTYYNPSLKSRFTISRDTSKNQYYLQLNSVTAEDTAVYYCTSERNWGAFAYWGQGTLVTVSS

SEQ ID NO: 47 - protein, 107 aa, synthetic construct
DILLTQSPSSLSATPGERATITCRASQGIRNNLNWYQQKPGQAPRLLIYYSSNLQSGVPSRFSGSGSGTDFTLTISRLQPEDVAAYYCQQSIKLPFTFGSGTKVEIK

SEQ ID NO: 48 - protein, 116 aa, synthetic construct
EVQLVESGAGVKQPGGTLRLTCTASGFSLTTYGVHWVRQAPGKGLEWVGVIWGDGRTDYDAAFMSRVTISKDTSKSTVYLQMNSLRAEDTAVYYCMRNRHDWFDYWGQGTTVTVSS

SEQ ID NO: 49 - DNA, 816 bp, synthetic construct
  source 1..816: mol_type = other DNA
atggagaccgacacactgctgctgtgggtgctgctgctgtgggtgccaggatccaccggcgacatcgtgatgacacagagcgcctccagcctgccagtgacccctggagagccagcttccatcagctgcagatctgacaagtccctgctgcacagcaacggcaatacatacctgtattggttcctgcagaagcccggccagtctcctcaggtgctgatctacagaatgagcaacctggcctctggagtgcctgaccgcttctctggatccggaagcggaaccgacttcaccctgacaatctctcgcgtggaggctgaggatgtgggcgtgtactattgcttccagcagctggagtacccatatacctttggccagggcacaaagctggagatcaagggaggaggaggatccggaggaggaggatctggcggcggcggctctcaggtgcagctggtgcagtccggagctgaggtggtgaagccaggagcttccgtgaagatcagctgtaaggcctctggcttcaatatcaaggacacctacatctattgggtgaggcagaggacaggacagggactggagtggatcggcaggatcgagccaatgaacggcaataccaagtacgatcccaagtttcagggccggctgaccatgacagccaacacctccaagaatacagcttatctgcagctgtcttccctgacctctgaggacacagccgtgtactattgttccaggggcggctggctgctgctgtacgtgatggattattggggccagggcaccacagtgacagtgagctctggcggcggcggcagc

SEQ ID NO: 50 - DNA, 819 bp, synthetic construct
  source 1..819: mol_type = other DNA
atggagcacagcaccttcctgtctggcctggtgctggccacactgctgtcccaggtgagccctgacatcgtgatgacccagtccgcttccagcctgcctgtgacacctggagagtccgtgagcatctcttgccggtctgataagtccctgctgcatagcaacggcaatacctacctgtattggtttctgcagaggccaggccagtctccccaggtgctgatctacaggatgtccaacctggccagcggagtgccagacagattctccggcagcggctctggcacagagtttaccctgacaatctctagagtggaggctgaggatgtgggcgtgtactattgcttccagcagctggagtacccttatacctttggcccaggcacaaagctggagatcaagggcggcggcggctctggaggaggaggatccggaggaggaggcagccaggtgcagctgctgcagtccggagctgaggtggtgaagccaggagcttctgtgaagatctcctgtaaggccagcggcttcaatatcaaggacacctacatctattgggtgaagcagccaacaggacagggcctggagtggatcggaaggatcgagcccatgaacggcaataccaagtacgatcctaagttccagggcaggtttaccatgacagccaacacctccatcaatacagcttatctgcagctgtcttccctgacctctgaggacacagccgtgtactattgttcccgcggaggatggctgctgctgtacgtgatggattattggggccagggcaccacagtgaccgtgagctctggcggcggcggcagc

SEQ ID NO: 51 - DNA, 819 bp, synthetic construct
  source 1..819: mol_type = other DNA
atggagcactccaccttcctgagcggcctggtgctggccacactgctgtctcaggtgtcccctgacatcgtgatgacccagtccccactgagcctgtctgtgacacccggcgagcctgcttccatcagctgcagaagcgataagtctctgctgcattccaacggcaatacctacctgtattggtttctgcagaagccaggccagagcccccaggtgctgatctacaggatgtctaacctggcctccggagtgcctgaccggttctctggatccggaagcggaaccgacttcaccctgaagatctccagggtggaggctgaggacgtgggcgtgtactattgcttccagcagctggagtacccatatacctttggccagggcacaaagctggagatcaagggaggaggaggaagcggaggaggaggatctggcggcggcggctcccaggtgcagctggtgcagtctggagctgagctggtgaagccaggagctagcgtgaagctgtcttgtaaggcctccggcttcaatatcaaggacacctacatctattgggtgaggcaggctccaggacagggcctggagtggatcggaaggatcgagcccatgaacggcaatacaaagtacgatcctaagtttcagggcagggtgaccatgacagccaacacctctatcaatacagcttatctggagctgtccagcctgagaagcgaggacaccgccgtgtactattgttctcgcggcggctggctgctgctgtacgtgatggattattggggccagggcaccctggtgacagtgtcttccggaggaggaggatcc

SEQ ID NO: 52 - DNA, 819 bp, synthetic construct
  source 1..819: mol_type = other DNA
atggagcactccaccttcctgagcggcctggtgctggccacactgctgtctcaggtgtcccccgacatcgtgatgacccagagcccatccagcctgtctgtgacaccaggagagagggtgtccatcagctgccggagcgataagtctctgctgcattctaacggcaatacctacctgtattggtttctgcagaagcccggccagtcccctcaggtgctgatctacagaatgtctaacctggcctccggagtgccagaccgcttctctggatccggaagcggcacagacttcaccctgacaatctccagggtggaggctgaggacgtgggcgtgtactattgcttccagcagctggagtacccctatacctttggccagggcacaaagctggagatcaagggaggaggaggatccggaggaggaggaagcggcggcggcggctctgaggtgcagctggtgcagtccggagctgagctggtgaagccaggagctagcgtgaagctgtcttgtgccgcttccggcttcaatatcaaggacacctacatctattgggtgagacaggctcctggacagggcctggagtgggtgggccgcatcgagcctatgaacggcaatacaaagtacgatccaaagttccagggcaggtttaccatgacagccaacaccagcaagaatacagcttatctgcagctgtcttccctgaggtccgaggacaccgccgtgtactattgtagccggggaggatggctgctgctgtacgtgatggattattggggccagggcaccctggtgacagtgagctctggaggaggaggatct

SEQ ID NO: 53 - DNA, 795 bp, synthetic construct
  source 1..795: mol_type = other DNA
atggagaccgacacactgctgctgtgggtgctgctgctgtgggtgccaggaagcaccggagagaacgtgctgacccagtctcctgccacactgagcgtgtctcccggcgagcctgtgaccctgaattgcagggcttccagctctgtgatcagcacatatctgcactggtaccagcagaagccaggacaggctcccaggctgtggatctattccaccagcaacctggcctctggcatccctgctcggttctctggatccggaagcggcacagagtacaccctgacaatctccagcgtggagtccgaggatgtggccgtgtactattgccagcagttcagcggctacccatatacctttggccagggcacaaagctggagatcaagggaggaggaggaagcggaggaggaggatctggcggcggcggctcccaggtgcagctgcaggagtccggacctgagctggtgaagccatcccagaccctgagcctgacctgtaaggtgtctggcgactccatcaccagcggctactggcattggatcaggcagtttccaggccggaagctggagtgggtgggctacatcaactatagcggctacacatactataatccctctctgaagtccagactgaccatctccagggacacatccaagaaccagtatttcctggagctgagaagcgtgacctctgacgatacagccgtgtactattgtacctctgagcgcaattggggcgcctttgcttactggggccagggcaccctgatcacagtgtctagcggcggcggcggctcc

SEQ ID NO: 54 - DNA, 816 bp, synthetic construct
  source 1..816: mol_type = other DNA
atggagcactccaccttcctgagcggcctggtgctggccacactgctgtcccaggtgagccctgagaacgtgctgacccagtctcctgctagcctgtccgtgtccccaggagagagagtgaccctgaattgccgcgcttccagctctgtgatctccacatacctgcattggtatcagcagaagccaggcaagccacccaggctgtggatctacagcacctctaacctggcctctggcatccctgctcggttctccggaagcggatctggcacagagtacaccctgacaatctccagcgtggagtccgaggactttgccgtgtactattgccagcagttcagcggctacccatatacctttggccagggcacaaagctggagatcaagggaggaggaggatccggaggaggaggaagcggcggcggcggctctcaggtgcagctgcaggagtccggcccagagctggtgaagccctctgagacactgtccctgacctgtaaggtgtctggcgactccatcaccagcggctactggcactggatcaggcagtttccaggccagaagctggagtacatgggctatatcaactacagcggctatacatactataatccctctctgaagtccaggctgaccatcagccgggatacatctaagaaccagtacttcctgaagctgagaagcgtgacctctgacgatacagccctgtactattgtaccagcgagcgcaattggggcgcctttgcttattggggccagggcaccctgatcacagtgtcttccggcggcggcggctctgatgtgcagctggtgcag

SEQ ID NO: 55 - DNA, 816 bp, synthetic construct
  source 1..816: mol_type = other DNA
atggagcactccaccttcctgagcggcctggtgctggccacactgctgtctcaggtgtcccctgagaacgtgctgacccagtctccagccacactgtccgcctctcctggagagagagccaccctgtcctgcagggcttccagctctgtgatcagcacatatctgcattggtaccagcagaagccaggccaggcccccaagctgtggatctattccaccagcaatctggccagcggcgtgccagctaggttctctggctccggcagcggcacagactacaccctgacaatctccagcgtggagccagaggattttgccgtgtactattgccagcagttctctggctacccctatacctttggccagggcacaaagctggagatcaagggaggaggaggatccggaggaggaggaagcggcggcggcggctctcaggtgcagctgcaggagtccggacctggcctggtgaagccatctgagacactgtccctgacctgtacagtgagcggcgactctatcacatccggctattggcactggatcaggcagccacctggcaagggcctggagtggatcggctacatcaactatagcggctacacctactataatcccagcctgaagtctaggttcaccatcagccgggacacatctaagaaccagtactatctgcagctgaattctgtgaccgccgaggatacagccgtgtactattgtacctccgagagaaactggggcgcctttgcttactggggccagggcaccctggtgacagtgtcttccggaggaggaggatccgatgtgcagctggtgcag

SEQ ID NO: 56 - DNA, 798 bp, synthetic construct
  source 1..798: mol_type = other DNA
atggagcacagcaccttcctgtctggcctggtgctggccacactgctgtcccaggtgagccctgagaacgtgctgacccagtcccctgctagcctgtctgcttccccaggagagagagtgaccatcacatgccgcgcctccagctctgtgatcagcacctatctgcattggtaccagcagaagcccggccaggctcctaagctgtggatctatagcacatctaatctggcctctggagtgccagctaggttctccggaagcggatctggaaccgactacaccctgacaatctccagcgtggagcccgaggattttgctacatactattgccagcagttctccggctacccttatacctttggccagggcacaaagctggagatcaagggaggaggaggaagcggaggaggaggatctggcggcggcggctccgaggtgcagctgcaggagagcggaccaggcctggtgaagccatccggcaccctgagcctgacatgtgccgtgtctggcgactccatcaccagcggctattggcactggatcaggcaggctccaggcaagggcctggagtgggtgggctacatcaactattctggctacacatactataatccctctctgaagtccaggttcaccatctctcgggacacatccaagaaccagtactatctgcagctgaatagcgtgaccgccgaggatacagccgtgtactattgtacctctgagagaaactggggcgcctttgcttactggggccagggcaccctggtgacagtgtcttccggaggaggaggatcc

SEQ ID NO: 57 - protein, 246 aa, synthetic construct
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSSGGGGSGGGGSGGGGSDIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 58 - protein, 489 aa, synthetic construct
DIQMTQSPSSLSASPGDRVTISCRASQDIGNFLTWYQQKPGQAPKLLIYYRSRLHSGVPSRFSGSGSGTDYTLTISSLEPEDFATYYCQQDNTLPWTFGQGTKVEIKGGGGSGGGGSGGGGSEVQLVESGGGLVQPGGSLRLSCAASGFTFTNYYMSWVRQAPGKGLEWVAFIRDKANGYTTEYSASVKGRFTISRDNSKNTLYLQMNSLRAEDTAVYYCARVLRYLGYNFDYWGQGTLVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 59 - protein, 24 aa, synthetic construct
GTWEVIGIEALTSDYLYYISNEYK

SEQ ID NO: 60 - protein, 738 aa, synthetic construct
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 61 - protein, 22 aa, synthetic construct
VKQTLNFDLLKLAGDVESNPGP

SEQ ID NO: 62 - protein, 249 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRLASS

SEQ ID NO: 63 - protein, 28 aa, synthetic construct
MKTPWKVLLGLLGAAALVTIITVPVVLL

SEQ ID NO: 64 - protein, 469 aa, synthetic construct
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDN

SEQ ID NO: 65 - protein, 269 aa, synthetic construct
SALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 66 - protein, 272 aa, synthetic construct
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYL

SEQ ID NO: 67 - protein, 209 aa, synthetic construct
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIE

SEQ ID NO: 68 - protein, 154 aa, synthetic construct
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPS

SEQ ID NO: 69 - protein, 219 aa, synthetic construct
DIVMTQSPSSLSVTPGERVSISCRSDKSLLHSNGNTYLYWFLQKPGQSPQVLIYRMSNLASGVPDRFSGSGSGTDFTLTISRVEAEDVGVYYCFQQLEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 450 aa, synthetic construct
EVQLVQSGAELVKPGASVKLSCAASGFNIKDTYIYWVRQAPGQGLEWVGRIEPMNGNTKYDPKFQGRFTMTANTSKNTAYLQLSSLRSEDTAVYYCSRGGWLLLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 214 aa, synthetic construct
DIQMTQSPSSLSASPGDRVTISCRASQDIGNFLTWYQQKPGQAPKLLIYYRSRLHSGVPSRFSGSGSGTDYTLTISSLEPEDFATYYCQQDNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 452 aa, synthetic construct
EVQLVESGGGLVQPGGSLRLSCAASGFTFTNYYMSWVRQAPGKGLEWVAFIRDKANGYTTEYSASVKGRFTISRDNSKNTLYLQMNSLRAEDTAVYYCARVLRYLGYNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 74 - protein, 18 aa, synthetic construct
KESGSVSSEQLAQFRSLD

SEQ ID NO: 75 - protein, 14 aa, synthetic construct
EGKSSGSGSESKST

SEQ ID NO: 76 - protein, 18 aa, synthetic construct
GSTSGGGSGGGSGGGGSS

SEQ ID NO: 77 - protein, 18 aa, synthetic construct
GSTSGSGKPGSGEGSTKG

(19) United States
(12) Patent Application Publication
(10) Pub. No.: US 2026/0356969 A1
(43) Pub. Date: Nov. 12, 2026

(54) CD26-TARGETED ANTIGEN-BINDING PROTEIN AND PHARMACEUTICAL USE THEREOF

(71) Applicant: ZONHON BIOPHARMA INSTITUTE, INC., Changzhou (CN)

(72) Inventors: Bruce yong MA, Changzhou, Jiangsu (CN); Peipei CAO, Changzhou, Jiangsu (CN); Chennan GE, Changzhou, Jiangsu (CN); Jianhua HANG, Changzhou, Jiangsu (CN); Tao ZHANG, Changzhou, Jiangsu (CN); Yu ZHUANG, Jiangsu, Changzhou (CN)

(21) Appl. No.: 19/475,335

(22) PCT Filed: Apr. 12, 2024
(86) PCT No.: PCT/CN2024/087392

§ 371 Date: Oct. 14, 2025

(30) Foreign Application Priority Data
Apr. 14, 2023 (CN) 202310400063.5

(51) Int. Cl. C07K 16/28; A61K 39/00; A61P 35/00; C12N 15/85

(52) U.S. Cl. CPC C07K 16/2896; A61P 35/00; C07K 16/2809; C12N 15/85; A61K 2039/505; C07K 2317/24; C07K 2317/31; C07K 2317/565; C07K 2317/622; C07K 2317/732; C07K 2317/92

(57) ABSTRACT

The present application relates to antigen-binding proteins binding to humanized CD26 and use of the antigen-binding proteins in preparation of a drug for treating high-expression CD26 tumors. The present application discloses for the first time that, compared to other murine antibodies and corresponding chimeric antibodies, or humanized antibodies, the antigen-binding proteins containing HCDR1 shown in SEQ ID NO: 1, HCDR2 shown in SEQ ID NO: 2, HCDR3 shown in SEQ ID NO: 3, LCDR1 shown in SEQ ID NO: 4, LCDR2 consisting of Arg, Met, and Ser, and LCDR3 shown in SEQ ID NO: 5 exhibit better antigen affinity as well as good safety and efficacy. Compared to existing first-line therapeutic regimens for related tumors, the CD26 antigen-binding proteins also demonstrate more prominent efficacy and safety.

### TECHNICAL FIELD

The present disclosure relates to an antibody or antigen-binding protein that binds to human CD26, and the use of the antibody or the antigen-binding protein in the preparation of a medicament for treating a tumor with high CD26 expression.

### BACKGROUND OF THE INVENTION

CD26 is a multifunctional type II transmembrane glycoprotein, and is also found in a soluble form in plasma. CD26 commonly exists as a homodimer, with each monomer consisting of 766 amino acid residues and a molecular mass of approximately 110 kDa. The amino acid residues of CD26 can be divided into five regions from the inside to the outside: the intracellular region (1-6), the transmembrane region (7-28), the highly glycosylated region (29-323), the cysteine-rich region (324-551), and the C-terminal catalytic domain (552-766). The three-dimensional structure of CD26 molecule is closely associated with its function. CD26 (also known as DPP4) inhibitors have been used clinically for the treatment of type 2 diabetes for over a decade. The expression of CD26 on the surface of various tumor cells, such as malignant mesothelioma, renal cancer, prostate cancer, lung cancer, is significantly increased. For these types of tumors with high CD26 expression, CD26 is a valuable therapeutic target (CD26/DPP4—a potential biomarker and target for cancer therapy, Pharmacology & Therapeutics (2019), 198:135-159).

At present, the most advanced human CD26-targeted anticancer drug under development is the monoclonal antibody YS110, developed by Y'S Therapeutics, which has completed Phase I/II clinical trials. However, analysis of existing literature indicates that YS110 has demonstrated relatively low activity in vivo and in vitro preclinical studies. For example, in the study “A humanized anti-CD26 monoclonal antibody inhibits cell growth of malignant mesothelioma via retarded G2/M cell cycle transition, Cancer Cell Int (2016) 16:35,” when tumor cells were treated with YS110 at a concentration of 250 μg/mL for 48 hours, the growth inhibition rate was 18.3%, and the IC50 value was significantly higher than 250 g/mL, indicating low activity of YS110 in vitro. Furthermore, the patent “Anti-CD26 Antibody and Methods of Use Thereof (Application No.: CN200680034937.4)” discloses therapeutic evaluations of YS110 in various mouse tumor models established with CD26 high-expressing cell lines, wherein YS110 only reduced tumor volume and alleviated tumor growth to some extent, but could not completely inhibit tumor growth to achieve tumor cure, suggesting the suboptimal activity of YS110 in animal models. The results of the completed Phase II clinical trial of YS110 showed good tolerability, but the disease control rate was below expectations, which is consistent with the low activity in its preclinical studies.

Although widespread expression of CD26 has been reported in the literature, YS110 has demonstrated a favorable safety profile, supporting its reliable safety in clinical use. In the Phase II clinical trial of YS110 involving 31 patients, no deaths occurred. The main side effects observed were infusion-related reactions (16.1%), hiccups (9.7%), and diarrhea (6.5%) (Phase 2 Study of YS110, a Recombinant Humanized Anti-CD26 Monoclonal Antibody, in Japanese Patients With Advanced Malignant Pleural Mesothelioma, JTO Clinical and Research Reports (2021), 2 (6)).

In summary, although the safety of CD26 as a therapeutic target has been validated in clinical trials, the existing CD26-targeting antibodies still face challenges in achieving satisfactory efficacy in tumor treatment.

Antigens play a crucial role in stimulating and initiating the immune response. An antigen is a substance that can stimulate the body to produce a specific immune response and can bind to immune response products such as antibodies or sensitized lymphocytes to elicit an immune effect. Antigens generally possess two intrinsic properties: immunogenicity and antigenicity. In practice, antigens do not function through their entire molecular structures; their function and specificity are generally achieved through epitopes. Immune cells initiate an immune response by recognizing and binding to specific epitopes on antigens.

Epitopes, also known as antigenic determinants, are specific sequences and spatial structures within an antigen molecule, composed of several amino acid residues, capable of eliciting an immune response. An antigen molecule contains multiple epitopes, including those that elicit protective immune responses, as well as some epitopes that are detrimental to protective immune responses, such as inhibitory or toxic epitopes. It is also recognized that among the various epitopes on a single antigen molecule, each contributes differently to antigenicity, leading to the distinction between dominant and non-dominant epitopes. Furthermore, factors such as the relative positions, conformational features, and flanking sequences of epitopes are closely related to their functional expression.

### SUMMARY OF THE INVENTION

To address the above problems, the present disclosure provides an isolated polypeptide, the amino acid sequence of which is identical to or has at least 90% sequence identity to the sequence as set forth in SEQ ID NO: 59 or SEQ ID NO: 64.

The present disclosure further provides a CD26 antibody or antigen-binding fragment with improved efficacy and safety profile. The CD26 antigen-binding protein of the present disclosure recognizes residues within the amino acid sequence as set forth in SEQ ID NO: 59 or SEQ ID NO: 64. As used herein, “recognizes the amino acid sequence region of CD26 as set forth in SEQ ID NO: 59 or SEQ ID NO: 64” refers to the ability to bind all or a portion of the residues within the amino acid sequences as set forth in SEQ ID NO: 59 or SEQ ID NO: 64 of CD26.

The CD26 antigen-binding protein of the present disclosure competes for binding to CD26 with an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5. Examples include the mouse antibody No. 79 or 19G300, as described in the embodiments.

The antigen-binding protein of the present disclosure recognizes the same CD26 fragment as recognized by an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5.

The antigen-binding protein of the present disclosure binds to residues within the amino acid sequence as set forth in SEQ ID NO: 64. As used herein, “binds to” refers to the ability to bind all or a portion of the residues within the amino acid sequence as set forth in SEQ ID NO: 64.

The antigen-binding protein of the present disclosure binds to residues within the amino acid sequence as set forth in SEQ ID NO: 59. As used herein, “binds to” refers to the ability to bind all or a portion of the residues within the amino acid sequence as set forth in SEQ ID NO: 59.

Methods known in the art can be used to determine whether a given antigen-binding protein recognizes the same CD26 fragment as an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5, or competes with said antigen-binding protein for binding to CD 26. For example, an antigen-binding protein having an amino acid sequence comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5 is allowed to bind to CD26 protein until saturation is achieved, and under this saturation condition, the binding ability of a given antigen-binding protein to CD26 protein is assessed. If the given antigen-binding protein is still able to bind to CD26 protein, it can be concluded that the given antigen-binding protein recognizes a distinct CD26 fragment from that recognized by the antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5. Conversely, if the given antigen-binding protein is unable to bind to CD26 protein under the saturation condition mentioned above, it can be concluded that the given antigen-binding protein competes for binding to the same CD26 fragment with the antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5. Further routine experiments, such as peptide mutation and binding analysis, can then be performed to determine whether the inability to bind to CD26 protein under the saturation condition is due to recognition of the same CD26 fragment or due to steric hindrance. For example, ELISA, RIA, Biacore, flow cytometry or any other quantitative or qualitative antibody-binding assay known in the art can be used.

Identification of the CD26 protein fragment recognized or bound by an antigen-binding protein can be performed using methods such as epitope mapping, X-ray crystallography, enzymatic digestion and chemical cleavage, synthetic peptides, phage display of random peptide libraries, alanine scanning, HDX-MS, and bioinformatics epitope prediction. In specific Examples 26 and 27 of the present disclosure, the binding of an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5, as well as another antigen-binding protein, to CD26 fragments was verified by enzymatic digestion of CD26 and by synthesizing CD26 protein fragments.

An antibody that competes with an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5 for binding to CD26, or that recognizes the same CD26 fragment, can be prepared using animal immunization methods as described in Example 1. Specifically, the CD26 fragment recognized by the antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5 is used as an immunogen to prepare CD26 monoclonal antibodies by existing methods. The antitumor efficacy and safety of the resulting antibodies are then evaluated through in vivo or in vitro experiments. In addition, other antibody preparation methods known in the art may also be employed.

The antigen-binding protein of the present disclosure is a monoclonal antibody, polyclonal antibody, recombinant antibody, human antibody, humanized antibody, chimeric antibody, multispecific antibody, or an antibody fragment thereof.

The antigen-binding protein of the present disclosure is a Fab fragment, Fab′ fragment, F(ab′)₂ fragment, Fv fragment, scFv, bispecific antibody, or a single-chain antibody.

The antigen-binding protein of the present disclosure is an antibody of the IgG1, IgG2, IgG3, or IgG4 subclass.

An isolated nucleic acid selected from any one of the following:
a) a DNA encoding an antigen-binding protein, wherein the antigen-binding protein is a CD26-binding antigen-binding protein as described above;
b) a DNA encoding a polypeptide, wherein the polypeptide has at least 90% sequence identity to SEQ ID NO: 59 or SEQ ID NO: 64;
c) a DNA that is fully complementary to the DNA of a) or b);
d) an RNA of a), b), or c), wherein U substitutes for T.

A vector comprising the nucleic acid described above.

A cell comprising the vector described above.

The nucleic acid molecule encoding the target antigen-binding protein can be administered using a viral or non-viral vector, which delivers the nucleic acid to a subject. Viral vectors that have been successfully used to deliver nucleic acids into host include retroviruses, adenoviruses, adeno-associated viruses (AAV), herpesviruses, poxviruses, and others.

A composition comprising any one of the antigen-binding proteins, nucleic acids, or cells described above.

A method for treating a disease associated with high-level CD26, comprising administering to a subject a therapeutically effective amount of any one of the antigen-binding proteins, nucleic acids, or cells described above. The disease associated with high-level CD26 includes, but is not limited to, a tumor, wherein the tumor with high-level CD26 includes, but is not limited to, renal cancer, mesothelioma, lung cancer, liver cancer, or prostate cancer. The antibody or antigen-binding fragment can be administered alone or in combination with other anticancer agents.

The present disclosure provides the following advantages over the prior art:

The present disclosure, for the first time, provides an antibody that competes with an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, Ser, and LCDR3 as set forth in SEQ ID NO: 5 for binding to CD26, or recognizes the same CD26 fragment as recognized by said antigen-binding protein, as well as an anti-CD26 antibody that binds to amino acid residues within the CD26 sequence as set forth in SEQ ID NO: 59 or SEQ ID NO: 64, which demonstrates improved antitumor efficacy and safety.

Specifically, the advantages include:

First, the antibody disclosed herein that binds to specific antigen fragments exhibits a significant efficacy advantage.

Data from Example 1 demonstrate that the mouse antibody No. 62 exhibits significantly higher lysis activity against human tumor cells compared with other mouse-derived antibodies, and its cytotoxicity against human tumor cells is comparable to that of the humanized antibody YS110, which has completed Phase II clinical trials.

Furthermore, data from Examples 5, 8, and 10 demonstrate that chimeric or humanized antibodies engineered based on the variable region of mouse antibody No. 62 exhibit superior activity in dot blot assays and higher antigen affinity compared with corresponding antibodies derived from other mouse antibodies. Example 11 demonstrates that, in vitro, BiTE antibodies derived from mouse antibody No. 62 induce PBMC cytotoxicity against multiple tumor cell types and exhibit superior cytotoxicity compared with the BiTE antibody wherein the CD26 antibody sequence is derived from YS110. Results from Examples 17 to 24 demonstrate that, in various animal tumor models, BiTE antibodies derived from mouse antibody No. 62 significantly inhibit tumor growth, and in some models, completely suppress tumor growth, showing superior efficacy compared with the BiTE antibody wherein the CD26 antibody sequence is derived from YS110. Data from Examples 23 and 24 further demonstrate that, in multiple animal tumor models, BiTE antibodies derived from mouse antibody No. 62 achieve significantly better tumor growth inhibition than existing first-line therapies, such as BAVENCIO for renal cancer.

Second, the antibody disclosed herein that binds to specific antigen fragments demonstrates a clear safety advantage.

Compared with BiTEs derived from other mouse antibodies, BiTEs engineered based on the variable region of mouse antibody No. 62 exhibit improved safety profiles (Example 13), with a lower risk of inducing cytokine release syndrome. Data from Example 12 show that anti-CD26 full-length IgG antibody cannot effectively induce T cell activation. The CD26-CD3 bispecific antibody in the BiTE format disclosed herein does not induce significant T cell activation prior to reaching the tumor site. T cell activation occurs only after the antibody binds to tumor cells, leading to activation of T cells around the tumor tissue and subsequent tumor cell killing, thereby enhancing therapeutic safety. Data from Examples 23 and 24 further demonstrate that the BiTE-format bispecific antibody of the present disclosure exhibit significantly improved safety compared with the full-length IgG antibody YS110 in various animal tumor models.

Moreover, the antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, Ser, and LCDR3 as set forth in SEQ ID NO: 5, such as the different humanized bispecific antibodies derived from mouse antibody No. 62 described in Example 10 (19G292, 19G293, 19G294), recognize and bind CD26 protein without being affected by the fusion of different anti-CD3 antibody segments or their binding to CD3 protein. The different humanized bispecific antibodies binding to different CD3 proteins (19G294, 18G272) exhibit the advantage of inducing lower levels of T cell-secreted cytokines (Example 13), while maintaining equivalent level of in vitro efficacy (Example 16). In vivo experiments have also demonstrated that bispecific antibodies binding to different CD3 proteins significantly inhibit tumor growth and exhibit improved safety (Example 22), further indicating that the safety and efficacy of the present disclosure are primarily attributed to the binding of CDR regions of the anti-CD26 antibody to CD26 protein fragment.

### BRIEF DESCRIPTION OF THE DRAWINGS

FIG. 1 is a dot blot analysis of humanized bispecific antibodies. FIG. 1a shows the dot blot activity analysis of humanized bispecific antibodies 18G272 and 18G278. FIG. 1b shows the dot blot activity analysis of humanized bispecific antibody 19G294.

FIG. 2 is a flow cytometry analysis showing the specificity of antibody binding to target cell antigens.

FIG. 3 shows the simultaneous binding of antibody 19G294 to CD26 protein and CD3 protein.

FIG. 4 shows the humanized bispecific antibody-mediated PBMC cytotoxic effect on PBMCs.

FIG. 5 shows the tumor volumes in a PC-3 cell subcutaneous xenograft model in NOD/SCID mice treated with antibody 18G272 or antibody 19G294.

FIG. 6 shows the animal body weights in a PC-3 cell subcutaneous xenograft model in NOD/SCID mice treated with antibody 18G272 or antibody 19G294.

FIG. 7 shows the tumor volumes in an NCI-H596 cell subcutaneous xenograft model in NOD/SCID mice treated with antibody 18G272.

FIG. 8 shows the tumor volumes in an NCI-H226 cell subcutaneous xenograft model in NOD/SCID mice treated with antibody 19G294.

FIG. 9 shows the animal body weights in an NCI-H226 cell subcutaneous xenograft model in NOD/SCID mice treated with antibody 19G294.

FIG. 10 shows the tumor volumes in an A498 cell subcutaneous xenograft model in NOD/SCID mice treated with antibody 19G294.

FIG. 11 shows the tumor volumes in an OS-RC-2 cell subcutaneous xenograft model in NOD/SCID mice treated with antibody 17G29 or antibody 19G295.

FIG. 12 shows the tumor volumes in an OS-RC-2 cell subcutaneous xenograft model in NOD/SCID mice treated with antibody 18G272 or 19G295.

FIG. 13 shows the animal survival rates in an OS-RC-2 cell subcutaneous xenograft model in NOD/SCID mice treated with antibody 18G272 or antibody 19G295.

FIG. 14 shows the tumor volumes in an OS-RC-2 cell subcutaneous xenograft model in NOD/SCID mice treated with antibody 18G272.

FIG. 15 shows the tumor volumes in an OS-RC-2 cell subcutaneous xenograft model in NOD/SCID mice treated with antibody 19G294.

FIG. 16 shows the tumor volumes in an OS-RC-2 cell subcutaneous xenograft model in NOD/SCID mice treated with different doses of antibody 19G294 or antibody 21G587.

FIG. 17 shows the animal body weights in an OS-RC-2 cell subcutaneous xenograft model in NOD/SCID mice treated with different doses of antibody 19G294 or antibody 21G587.

FIG. 18 shows the tumor volumes in an OS-RC-2 cell subcutaneous xenograft model in NOD/SCID mice treated with antibody 19G294, BAVENCIO in combination with axitinib or other agents.

FIG. 19 shows the tumor volumes on day 26 post-inoculation in an OS-RC-2 cell subcutaneous xenograft model in NOD/SCID mice treated with antibody 19G294, BAVENCIO in combination with axitinib, or other agents.

FIG. 20 shows the tumor volumes on day 26 post-inoculation in an A498 cell subcutaneous xenograft model in NOD/SCID mice treated with antibody 19G294, BAVENCIO in combination with axitinib, or other agents.

FIG. 21 shows the tumor weights on day 65 post-inoculation in an A498 cell subcutaneous xenograft model in NOD/SCID mice treated with antibody 19G294, BAVENCIO in combination with axitinib, or other agents.

FIG. 22 shows a TIC of amino acid sequence coverage analysis of trypsin-digested samples.

FIG. 23 shows an amino acid sequence coverage map of trypsin-digested samples.

FIG. 24 shows the structural schematic diagram of the CD26 fragment display vector.

FIG. 25 shows the binding of 19G294 in CD26 full extracellular domain and 21G572 display cells.

FIG. 26 shows the binding of 21G428-429 in 21G572 and other truncated CD26 fragment display cells.

FIG. 27 shows the differences in antibody binding activity in a mixed cell system as detected by flow cytometry.

FIG. 28 shows the competitive binding of 19G294 and 19G300 to CD26.

FIG. 29 shows the competitive binding of 19G294 and 17G29 to CD26.

### DETAILED DESCRIPTION OF THE INVENTION

Unless otherwise defined herein, the technical terms used in the present disclosure shall have the meanings commonly understood by those of ordinary skill in the art.

As used herein, singular forms such as “a,” “an,” and “the” include their plural referents unless the context clearly dictates otherwise.

The term “or” as used herein is intended to mean “and/or” and may be used interchangeably with “and/or.”

The term “CD26” is also referred to as dipeptidyl peptidase 4 (DPP4). The amino acid sequence of human CD26 is available in the GenBank database under the accession number NP_001926.2, and its cDNA sequence is available in the GenBank database under the accession number NM 001935.3.

The term “conservative amino acid substitution” refers to the replacement of an original amino acid with another amino acid that essentially does not alter the chemical, physical, and/or functional properties of an antibody or a fragment, such as binding affinity to CD26. Conservative substitutions of amino acids are well known in the art. For example, alanine (Ala) can be conservatively substituted with glycine (Gly) or serine (Ser); arginine (Arg) can be substituted with lysine (Lys) or histidine (His); asparagine (Asn) can be substituted with glutamine (Gln) or histidine (His), etc.

The term “affinity” refers to the strength of the interaction between an antibody and an antigen. The variable region of an antibody interacts with an antigen through noncovalent forces; the more interactions, the higher the affinity.

The term “antibody” refers to a member of the immunoglobulin family capable of binding a corresponding antigen in a specific, noncovalent, and reversible manner. For example, a naturally occurring IgG antibody is a tetramer comprising at least two heavy chains and two light chains linked to each other via disulfide bonds. Each heavy chain comprises a heavy chain variable region (VH) and a heavy chain constant region (CH). The heavy chain constant region comprises three domains: CH1, CH2, and CH3. Each light chain comprises a light chain variable region (VL) and a light chain constant region (CL). The light chain constant region comprises a single CL domain. The VH and VL regions can be further subdivided into complementarity-determining regions (CDRs), also referred to as hypervariable regions, and more conserved framework regions (FRs). Each VH and VL comprises three CDRs and four FRs arranged in the following order from the amino-terminus to the carboxyl-terminus: FR1, CDR1, FR2, CDR2, FR3, CDR3, and FR4. HCDR1, HCDR2, and HCDR3 refer to the three heavy chain complementarity-determining regions; LCDR1, LCDR2, and LCDR3 refer to the three light chain complementarity-determining regions. The positions of the CDRs and FRs can be defined using various numbering schemes known in the art, such as Kabat, Chothia, and IMGT. The present disclosure adopts the IMGT numbering scheme. The variable regions of the heavy chain (VH) and light chain (VL) are responsible for antigen recognition, particularly through their complementarity-determining regions (CDRs), which together confer specificity to particular epitopes on the antigen. The constant regions are primarily responsible for effector functions.

The term “antigen-binding fragment” refers to a fragment of an antibody that retains the ability to specifically bind to an antigen, for example, a fragment that retains one or more complementarity-determining regions (CDRs). Examples include, but are not limited to, Fab fragment, Fv fragment, bispecific antibody, linear antibody, single-chain antibody, nanobody, and multispecific antibody.

The term “monoclonal antibody” refers to a population of antibodies that is essentially homogeneous, meaning that, except for minor naturally occurring mutations, the antibody molecules within the population have identical amino acid sequences. Polyclonal antibodies, by contrast, generally comprise a variety of different antibodies with different amino acid sequences in their variable domains, particularly in their complementarity-determining regions (CDRs). Monoclonal antibodies can be obtained using methods known to those skilled in the art. In the present disclosure, monoclonal antibodies are obtained using hybridoma technology, which is one of the known methods of monoclonal antibody production.

The term “murine antibody” refers to an antibody consisting only of a rat or mouse immunoglobulin sequence.

The term “chimeric antibody” refers to an antibody in which the variable regions are derived from a non-human (e.g., murine) antibody sequence, and the remaining portions are derived from a human antibody sequence.

The term “humanized antibody” refers to an antibody in which the CDRs are derived from a non-human (e.g., murine) antibody sequence, and the remaining portions are derived from a human antibody sequence.

The term “bispecific antibody” refers to an antibody having two antigen-binding sites with specificity for the same or different antigens.

The term “BiTE” refers to a type of bispecific antibody known as a “bispecific T cell engager”. It is composed of two antigen-specific single-chain variable fragments (scFvs) connected via a linker, wherein one scFv is specific for a tumor-associated surface antigen and the other scFv is specific for CD3 expressed on the surface of T cells. A single-chain variable fragment (scFv) is composed of a heavy chain variable region and a light chain variable region connected via a linker. The “linker” refers to an amino acid sequence used to join different protein fragments. Numerous studies have been conducted on the design and selection of linkers. In a BiTE, a linker connects a heavy chain variable region and a light chain variable region to form a scFv, and another linker connects two scFvs in tandem and enables their free rotation. Such linkers are generally composed of repeated GGGGS sequences, where S denotes serine and G denotes glycine. Glycine, with its small molecular weight and short side chain, contributes to the flexibility of the side chain, while serine, due to its strong hydrophilicity, enhances the hydrophilicity of the peptide chain. Optimizing the number of repeat sequences can not only facilitate the correct conformational association between the light chain and heavy chain in the scFv, but also influence the distance between the two scFvs to promote optimal interactions between T cells and target cells at the immunological synapse. (Zhou Chong et al., Research Progress on Bispecific Single-Chain Antibody BITE [J], Journal of Biology, 2018). Linkers commonly used to connect the heavy chain variable region and the light chain variable region in scFvs include, for example, KESGSVSSEQLAQFRSLD, EGKSSGSGSESKST, GSTSGGGSGGGSGGGGSS (US20180326032), and GSTSGSGKPGSGEGSTKG (Preclinical Development of Bivalent Chimeric Antigen Receptors Targeting Both CD19 and CD22). In the specific embodiments of the present disclosure, the flexible linker connecting a heavy chain and a light chain to form an scFv comprises three repeats of the GGGGS motif, and the flexible linker connecting two scFvs in tandem comprises a single GGGGS motif. One of ordinary skill in the art can select and determine linkers from known linkers based on the teachings of the prior art through routine experimentation and limited experiments. The number of GGGGS repeats in the two types of flexible linkers can be optimized, and flexible linkers other than GGGGS can also be used.

CD3 is a component of T cell signal transduction. When a BiTE molecule simultaneously binds to a T cell and a tumor cell, the T cell is activated, promoting CD8+ T cells to directly secrete perforin and granzyme, and CD4+ T cells to secrete cytokines that further recruit and activate cytotoxic T cells, thereby killing tumor cells. The anti-CD3 antibody sequence can be selected from OKT-3, L2K, TR66, UCHT1, SP34, IORT3, Catumaxomab, Blinatumomab, Solitomab, WBP3311_2.306.4 (patent CN201880061333.1), as well as other anti-CD3 antibody sequences known in the art. In the specific embodiments of the present disclosure, the anti-CD3 antibody sequences from Solitomab and WBP3311_2.306.4 were used. A person skilled in the art can select and determine other known anti-CD3 antibody sequences through routine experimentation and limited experiments, and is not restricted to the specific anti-CD3 antibody sequence exemplified herein.

The term “chimeric bispecific BiTE antibody” as used herein refers to a murine-derived CD26 scFv linked to a humanized CD3 scFv by a linker.

The terms “administration” or “administering”, and “treatment” or “treating”, refer to the contact of an exogenous agent, therapeutic agent, or diagnostic agent with the tissues, organs, cells, or biological fluids of a subject. The term “treatment” or “treating” refers to delaying or preventing the development of clinical symptoms of a disease, alleviating or improving at least one physical parameter, or preventing the progression of the disease.

### Example 1. Preparation and Screening of Hybridoma Cell Lines Against Human CD26

### Step 1: Animal Immunization

Based on the publicly available cDNA sequence of CD26 (GenBank accession number: NM_001935.3), a recombinant CD26 (rCD26) expression vector was designed. A histidine tag was introduced at the 5′ end of the codon, and the full-length gene was synthesized. The synthesized gene was cloned into the pCHO1.0 plasmid, expressed in CHO-S host cells, and subsequently purified. The prepared recombinant CD26 protein was used to immunize female BALB/c mice following routine immunization procedures. Detailed immunization protocols are described in MAKING and USING ANTIBODIES: A PRACTICAL HANDBOOK. Indirect ELISA was employed to monitor the serum titers of immunized mice. The mouse exhibiting the highest serum titer was selected, and its spleen cells were fused with myeloma cells.

### Step 2: Cell Fusion

### (1) Preparation of Spleen Cells

Orbital blood was collected from the immunized mouse, which was then sacrificed by cervical dislocation and immersed in 75% (v/v) ethanol for 10 minutes. The spleen was aseptically harvested on a sterile workbench, placed on a cell strainer, thoroughly dissociated, and then passed through the cell strainer. The cells were washed several times by centrifugation with sterile 1640 medium (purchased from Gibco), and subsequently resuspended to obtain a single-cell suspension. The cells were counted and retained for subsequent use.

### (2) Preparation of Feeder Cells

A female BALB/c mouse aged 8-10 weeks was selected. Orbital blood was collected to obtain negative serum. The mouse was sacrificed by cervical dislocation and then immersed in 75% (v/v) ethanol for 10 minutes. Under aseptic conditions, the abdominal skin was incised to expose the peritoneum. Approximately 10 mL of 1640HT medium (purchased from SIGMA) was injected into the peritoneal cavity using a syringe. The abdomen was gently massaged, and the fluid was aspirated and re-injected several times. The medium containing macrophages was aspirated and added to 20% 1640HAT medium for later use.

A female BALB/c mouse aged 2-3 weeks was selected. The mouse was sacrificed by cervical dislocation and then immersed in 75% (v/v) ethanol for 10 minutes. Under aseptic conditions, the thymus was harvested and placed on a cell strainer, dissociated and passed through the cell strainer. The resulting thymocytes were added to the above 20% 1640HAT medium containing macrophages for subsequent use.

### (3) Cell Fusion

The mouse myeloma cell line SP2/0 in logarithmic growth phase was collected and counted. Approximately 1×108 spleen cells and 2×107 SP2/0 cells (as described above) were added to a fusion tube, mixed, and centrifuged at 1,000 rpm for 10 minutes. The supernatant was discarded, and the fusion tube was placed in the palm and gently rocked back and forth to loosen the pellet. Within 60 seconds, 1 mL of pre-warmed PEG 1450 (polyethylene glycol 1450; purchased from SIGMA) was added slowly at first and then gradually faster, followed by adding 30 mL of 1640HT medium to terminate the reaction. The mixture was centrifuged at 1,000 rpm for 10 minutes; the supernatant was discarded, and the pellet was loosened by rubbed gently and added to the 20% 1640HAT medium obtained in Step 2.

After thorough mixing, the above HAT medium was dispensed into a 96-well cell culture plate at 200 μL per well and incubated at 37 °C in a 5% CO₂ incubator. After one week, the 20% 1640HAT medium was replaced with 10% 1640HT medium, and the supernatant was collected for assay three days later.

### Step 3: Screening of Hybridoma Cell Lines Specific for CD26

(1) Preparation of ELISA plate: The recombinant CD26 target protein was diluted to 1 μg/mL using CB coating buffer and coated onto a 96-well ELISA plate at 100 μL per well. The plate was incubated at 2-8 °C overnight. After washing once, the plate was tapped dry. The wells were blocked with PBST buffer containing 2% BSA (200 μL per well) at 37 °C for 2 hours. The plate was then tapped dry and kept for later use.
(2) Screening of positive clones: The culture supernatant of the cells to be tested was added to the prepared ELISA plate (100 μL per well) and incubated at 37 °C for 30 minutes. After washing, the plate was tapped dry. Diluted HRP-conjugated goat anti-mouse IgG was added at 100 μL per well and incubated at 37 °C for 30 minutes. After washing, the plate was tapped dry. TMB chromogenic solution was added at 100 μL per well and incubated at 37 °C in the dark for 15 minutes. Then, 50 μL per well of 2 M H₂SO₄ was added to terminate the reaction. The absorbance at 450 nm (OD₄₅₀) was measured. A well was determined to be positive if the (OD₄₅₀ value/negative control value) was ≥2.1. Positive clones were selected for clonal screening. After three to four rounds of clonal screening, a monoclonal cell line with a 100% positivity rate was confirmed as a stable cell line, and the cell line was established.

### Step 4: Activity Evaluation of Mouse Anti-CD26 Monoclonal Antibody

In the 786-0 cell assay system, 786-0 cells were labeled with a green fluorescent signal using Calcein-AM fluorescent dye. The 786-0 cells were seeded into a U-bottom 96-well cell culture plate at a concentration of 6×10⁵ cells/mL (50 μL per well). Three replicate wells were set for each group. 50 μL of anti-CD26 antibody was added to the corresponding sample wells at a final concentration of 100 ng/mL. For the blank control wells, 50 μL of culture medium was added; for the positive control wells, 50 μL of Triton X-100 at a final concentration of 1% was added. The plate was incubated at 37 °C for 30 minutes. Subsequently, PBMCs (peripheral blood mononuclear cells) at a concentration of 6×10⁶ cells/mL were added at an effector-to-target (E:T) ratio of 10:1. The reaction system was incubated at 37 °C under CO₂ atmosphere for 5 hours. After incubation, the plate was centrifuged, and the supernatant was collected and transferred to a new 96-well plate. After a second centrifugation, 80 μL of supernatant was collected and transferred to a black 96-well plate. Fluorescence intensity was measured using a microplate reader at 470 nm excitation wavelength and 515 nm emission wavelength.

The formula for calculating the cell lysis rate was as follows:

Cell lysis rate (%)=[(Vsample−Vvehicle control)/(VTriton X-100−Vvehicle control)]×100%

(Vsample is the mean value of the fluorescence signal reading of the drug-treated group measured at the excitation and emission wavelengths. Vvehicle control is the mean value of the fluorescence signal reading of the blank control group measured at the excitation and emission wavelengths. VTriton X-100 is the mean value of the fluorescence signal reading of the positive control group measured at the excitation and emission wavelengths.)

TABLE 1
Antibody-mediated PBMC cytotoxic effect against 786-0 target cells

| Antibody No. | 72 | 160 | 62 | 51 | 212 | 79 | YS110 |
|---|---|---|---|---|---|---|---|
| Cell lysis rate | 8.6% | 13.0% | 53.1% | 12.3% | 11.3% | 48.8% | 56.6% |

From the above results, under the conditions of 100 ng/mL antibody concentration and 5 hours of incubation, mouse antibody No. 62 mediated PBMC cytotoxicity against 786-0 target cells resulting in 53.1% cell lysis rate, and mouse antibody No. 79 mediated PBMC cytotoxicity against 786-0 target cells resulting in 48.8% cell lysis rate. The cytolytic activity of antibodies No. 62 and No. 79 against 786-0 target cells was significantly higher than that of other mouse monoclonal antibodies (Nos. 72, 160, 51, 212), and was comparable to the humanized antibody YS110.

786-0 is a human-derived tumor cell line. Theoretically, a successfully humanized antibody exhibits much higher affinity for human cells than its parental murine antibody, and therefore exerts stronger lytic activity against such tumor cells. The cytotoxicity of mouse antibodies No. 62 and No. 79 against human tumor cells was already comparable to that of the humanized antibody YS110, which has completed Phase II clinical trials, demonstrating the advantages of the mouse antibodies No. 62 and No. 79. It can be reasonably expected by those skilled in the art that humanized antibodies derived from parental antibodies No. 62 and No. 79 would exhibit superior cytotoxicity against human tumor cells compared with mouse antibodies No. 62 and No. 79, as well as compared with YS110, which has been further confirmed in Example 11.

### Example 2. Sequencing and Screening of Variable Region Sequences from Hybridoma Cell Lines

Step 1. Total RNA Extraction from CD26-Specific Hybridoma Cells

The hybridoma cells were passaged into T75 culture flasks and cultured until approximately 90% confluence. The cells were then digested and collected by centrifugation. Total RNA was extracted from the monoclonal hybridoma cell lines using an RNA extraction kit (purchased from Roche). The extracted total RNA was used as a template for first-strand cDNA synthesis via reverse transcription using a cDNA synthesis kit (purchased from Thermo). The reaction products were stored at −20 °C; and for long-term storage, were stored at −70 °C.

### Step 2. PCR Amplification of Heavy and Light Chain Variable Region Genes

The first-strand cDNA of the hybridoma cells was used as a template, and 1 μL of cDNA, 5 μL of 10×PCR buffer, 1 μL each of forward and reverse primers (25 μmol), 1 μL of dNTPs, 1 μL of 25 mmol/L MgCl₂, and 39 μL of HLO were added respectively to form a 50 μL reaction system. After initial denaturation at 95 °C for 10 minutes, 1 μL of Taq polymerase was added to initiate thermal cycling for PCR amplification. The reaction conditions were as follows: denaturation at 94 °C for 1 minute, annealing at 58 °C for 1 minute, and extension at 72 °C for 1.5 minutes, for a total of 30 cycles, followed by holding at 72 °C for 10 minutes. 5 μL of the PCR product was analyzed by 1.2% agarose gel electrophoresis.

### Step 3. Cloning and Sequencing of Heavy and Light Chain Variable Region Genes

According to the instructions of the pGM-T Fast Ligation Kit (VT207-02, Beijing Tiangen Biotech Co., Ltd.), the variable region genes of the heavy and light chains were respectively ligated into the pGM-T vector and transformed into Escherichia coli Top10 competent cells. Blue-white screening was performed, and the transformants were cultured at 37 °C for 12-16 hours.

The resulting white colonies were inoculated into 1-5 mL of LB medium containing ampicillin at a final concentration of 100 μL, and shaken at 37 °C for 3-4 hours. PCR was performed to identify clones carrying the correct insert. The positive clones identified by PCR screening were subjected to sequencing. The sequencing results were analyzed by comparison with the IMGT database to determine the amino acid sequences of the variable regions and complementarity-determining regions (CDRs) of the heavy and light chains of the antibody.

The monoclonal mouse antibody No. 62 comprises HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4; LCDR2 consisting of Arg, Met, Ser (where Arg represents arginine, Met represents methionine, and Ser represents serine), and LCDR3 as set forth in SEQ ID NO: 5. The heavy chain variable region is shown in SEQ ID NO: 31, and the light chain variable region is shown in SEQ ID NO: 32.

### Example 3. Design of Chimeric Bispecific BiTE Antibody

Using the heavy and light chain sequences of mouse monoclonal antibodies Nos. 72, 160, 62, and 51 obtained in Example 1 and sequenced according to the method of Example 2, bispecific antibodies simultaneously targeting CD26 and CD3 were designed.

The variable region of the light chain targeting CD26 was connected to the variable region of the heavy chain targeting CD26 via a short linker peptide (e.g., SEQ ID NO: 12) to form a single-chain anti-CD26 antibody domain. These single-chain antibodies are: single-chain antibody derived from mouse antibody No. 72 (e.g., SEQ ID NO: 16), single-chain antibody derived from mouse antibody 160 (e.g., SEQ ID NO: 17), single-chain antibody derived from mouse antibody 62 (e.g., SEQ ID NO: 18), and single-chain antibody derived from mouse antibody 51 (e.g., SEQ ID NO: 19).

The sequence of the single-chain antibody targeting CD3 is formed by connecting the variable region of the heavy chain (e.g., SEQ ID NO: 10) and the variable region of the light chain (e.g., SEQ ID NO: 11) via a short linker peptide (e.g., SEQ ID NO: 12).

Four first-recognition domain sequences were respectively fused with the second-recognition domain sequence via a short linker peptide (e.g., SEQ ID NO: 13) to form four BiTE-structured bispecific antibodies. The four BiTEs are: 17G105 derived from mouse antibody No. 72 (e.g., SEQ ID NO: 20), 17G108 derived from mouse antibody No. 160 (e.g., SEQ ID NO: 21), 17G61 derived from mouse antibody No. 62 (e.g., SEQ ID NO: 22), and 17G131 derived from mouse antibody No. 51 (e.g., SEQ ID NO: 23).

### Example 4. Expression Plasmid Construction, Expression, and Purification of Anti-CD26-CD3 Chimeric Bispecific Antibody

The murine (Mus musculus) IgG k signal peptide sequence (e.g., SEQ ID NO: 24) was added upstream of the above CD26 single-chain antibody amino acid sequences (SEQ ID NOs: 16-19), and the sequences were optimized based on the codon preference of mammalian CHO cells to generate the optimized CD26 single-chain antibody gene sequences (SEQ ID NOs: 25-28). An AvrII restriction site and a Kozak sequence were introduced upstream of the optimized sequences. The Solitomab CD3 single-chain antibody amino acid sequence was codon-optimized to generate the optimized gene sequence (SEQ ID NO: 29), and a linker peptide gene (e.g., SEQ ID NO: 30) was added upstream of the gene, and a stop codon together with a BstZ17I restriction site were added downstream of the gene. These optimized AvrII restriction site, Kozak sequence, signal peptide, and CD26 single-chain antibody sequences were fused via a linker peptide gene with the CD3 single-chain antibody, stop codon, and BstZ17I restriction site to generate four chimeric BiTE genes. The resulting genes were synthesized and constructed into pUC57 plasmids, producing long-term storage plasmids, which were designated as pUC57-17G105, pUC57-17G108, pUC57-17G61, and pUC57-17G131.

The target BiTE genes were amplified by PCR, and the PCR products were purified from 1% agarose gel. The purified PCR products and the pZHK 2.0 vectors were double-digested with AvrII and BstZ17I. The pCHO 1.0 backbone vectors purchased from Invitrogen were digested with SfiI to remove “Expression Frame 1” and subsequently circularized using T4 DNA ligase to generate the pZHK 2.0 vectors.

The double-digested PCR products were ligated into the pZHK 2.0 vectors using T4 DNA ligase and transformed into Top10 competent cells. The transformants were plated on LB plates containing kanamycin and incubated overnight at 37 °C. Positive clones were screened on the following day and sequenced, and the obtained sequences were fully consistent with the expected sequences.

Mammalian cell lines stably expressing high levels of the CD26-CD3 chimeric bispecific BiTE antibodies were inoculated into Dynamis medium (A2617501, purchased from Thermo Fisher Scientific) and cultured in a fed-batch mode at 37 °C, 8% CO₂, and 130 rpm. The culture supernatants were harvested and subjected to low-temperature centrifugation at 12,000 rpm for 15 minutes to collect the supernatants, followed by filtration through a 0.45 μm membrane to obtain the clarified culture supernatants. The clarified supernatants were subjected to chromatographic purification. The resulting products exhibited an approximate molecular weight of 55 kDa, consistent with the correct size.

### Example 5. Affinity Analysis of Anti-CD26-CD3 Chimeric Bispecific Antibody for CD26 Protein

The binding affinity of the chimeric antibodies for antigen protein CD26 were measured using the ForteBio Octet QKe instrument for biomolecular interaction analysis. First, the amine-reactive biosensor AR2G was activated with EDC/s-NHS. The activated AR2G biosensor was then immersed in a CD26 protein solution diluted with 10 mM sodium acetate (pH 5.0) solution for immobilization. The biosensors with immobilized CD26 protein were quenched in 1 M ethanolamine solution (pH 8.5), followed by equilibration in 1× kinetics buffer. After equilibration, the biosensors were exposed to the humanized bispecific antibody solutions for association, and subsequently placed in 1× kinetics buffer for dissociation. Data analysis was performed using ForteBio Data Analysis 8.0 software to calculate the affinity constant (KD), the association rate constant (kon), and the dissociation rate constant (kdis). The response value represents the number of antibody molecules that can bind to the antigen; kon represents the binding rate of the antibody to the corresponding receptor; kdis represents the dissociation rate of the antibody from the corresponding receptor; NA indicates no valid data.

TABLE 2
Affinity analysis of the chimeric antibody for CD26 Protein

| | assay | | | | |
|---|---|---|---|---|---|
| Sample | concentration | | KD | kon | kdis |
| No. | (nM) | Response | (M) | (1/Ms) | (1/s) |
| 17G61 | 181.8 | 0.8493 | 1.73E−08 | 1.91E+04 | 3.31E−04 |
| 17G105 | 181.8 | 0.4979 | 3.93E−08 | 3.20E+04 | 1.26E−03 |
| 17G131 | 181.8 | 0.1496 | 4.36E−08 | 1.90E+04 | 8.29E−04 |
| 17G108 | 181.8 | 0.0395 | NA | NA | NA |

From the above data, it is evident that the chimeric antibodies are capable of binding to and dissociating from CD26 protein. 17G61 (anti-CD26-CD3 chimeric bispecific antibody derived from mouse antibody No. 62) exhibited the highest response value with CD26 protein, indicating that, under the same concentration conditions, a greater number of 17G61 molecules bound to CD26 protein. In contrast, 17G105 (anti-CD26-CD3 chimeric bispecific antibody derived from mouse antibody No. 72) and 17G131 (anti-CD26-CD3 chimeric bispecific antibody derived from mouse antibody No. 51) showed significantly lower response values upon binding to CD26 protein compared with 17G61. Furthermore, 17G61 demonstrated a smaller KD value with CD26, indicating that 17G61 possesses a higher binding affinity for CD26 protein.

### Example 6. Humanization of Anti-CD26 Mouse Antibody

Humanization was performed based on the sequences of candidate mouse antibodies No. 62 and 212 from Example 1, which exhibited high affinity for CD26 and strong cytotoxic activity against CD26 high-expressing tumor cells. The variable regions of the heavy chain (SEQ ID NO: 31) and light chain (SEQ ID NO: 32) of mouse antibody No. 62, as well as the variable regions of the heavy chain (SEQ ID NO: 33) and light chain (SEQ ID NO: 34) of mouse antibody No. 212, were analyzed for humanization. Multiple humanized heavy chain and light chain combinations of each antibody were generated by computational predictions. Four distinct humanized heavy-light chain amino acid sequences of mouse antibody No. 62 were obtained: 18G272-VL (SEQ ID NO: 7), 18G272-VH (SEQ ID NO: 6), 19G292-VL (SEQ ID NO: 35), 19G292-VH (SEQ ID NO: 36), 19G293-VL (SEQ ID NO: 37), 19G293-VH (SEQ ID NO: 38), 19G294-VL (SEQ ID NO: 9), and 19G294-VH (SEQ ID NO: 8). The fully humanized antibody derived from mouse antibody No. 62 is designated as 21G428-429, with the heavy chain amino acid sequence as set forth in SEQ ID NO: 70 and the light chain amino acid sequence as set forth in SEQ ID NO: 69. The fully humanized antibody derived from mouse antibody No. 79 is designated as 21G430-431, with the heavy chain sequence as set forth in SEQ ID NO: 72 and the light chain sequence as set forth in SEQ ID NO: 71.

Four distinct humanized heavy-light chain amino acid sequences of mouse antibody No. 212 were obtained: 18G278-VL (SEQ ID NO: 39), 18G278-VH (SEQ ID NO: 40), 19G295-VL (SEQ ID NO: 41), 19G295-VH (SEQ ID NO: 42), 19G296-VL (SEQ ID NO: 43), 19G296-VH (SEQ ID NO: 44), 19G297-VL (SEQ ID NO: 45), and 19G297-VH (SEQ ID NO: 46).

The humanized antibody YS110 was used as a positive control, with its heavy chain and light chain designated as YS110-VH (SEQ ID NO: 48) and YS110-VL (SEQ ID NO: 47), respectively.

### Example 7. Expression Plasmid Construction, Stable Expression, and Purification of Humanized Bispecific Antibody

The light chain and heavy chain of each humanized CD26 antibody (18G272, 19G292, 19G293, 19G294, 18G278, 19G295, 19G296, 19G297) were linked via a short peptide linker (SEQ ID NO: 12), and a short peptide linker (SEQ ID NO: 13) was added downstream. The resulting sequences were optimized based on the codon preference of mammalian CHO cells to generate the optimized CD26 single-chain antibody gene sequences (SEQ ID NO: 49-56). An AvrII restriction site and a Kozak sequence were introduced upstream, and the CD3 single-chain antibody gene (SEQ ID NO: 29), a stop codon, and a BstZ17I restriction site were added downstream. The resulting humanized CD26-CD3 BiTE gene sequences were synthesized and cloned into the pUC57 plasmid, designated as pUC57-18G272, pUC57-19G292, pUC57-19G293, pUC57-19G294, pUC57-18G278, pUC57-19G295, pUC57-19G296, and pUC57-19G297.

Similarly, the light chain and heavy chain of antibody YS110 were linked via a short peptide linker (SEQ ID NO: 12), and a short peptide linker (SEQ ID NO: 13) was added downstream. An AvrII restriction site and a Kozak sequence were introduced upstream, and the CD3 single-chain antibody gene (SEQ ID NO: 29), a stop codon, and a BstZ17I restriction site were added downstream. The resulting humanized CD26-CD3 BiTE gene sequences was synthesized and cloned into the pUC57 plasmid, designated as pUC-17G29.

Additionally, the light chain and heavy chain of 19G294 were linked via a short peptide linker (SEQ ID NO: 12), and a short peptide linker (SEQ ID NO: 13) was added downstream. This construct was linked to a single-chain antibody (SEQ ID NO: 57) formed by linking the heavy and light chains of the CD3 antibody WBP3311_2.306.4 via a short peptide linker (SEQ ID NO: 12). The resulting sequence was optimized based on the codon preference of CHO cells. An AvrII restriction site and a Kozak sequence were introduced upstream, and a stop codon and a BstZ17I restriction site were added downstream. The resulting gene was synthesized and cloned into the pUC57 plasmid, designated as pUC57-21G587.

The target genes were amplified by PCR, and the PCR products were purified from 1% agarose gel. The purified PCR products and the pZHK2.0 vectors were double-digested with AvrII and BstZ17I. The double-digested products were ligated into pZHK2.0 using T4 DNA ligase, followed by transformation into Top10 competent cells. The transformants were plated on LB plates containing kanamycin and incubated overnight at 37 °C. Positive clones were screened and sequenced on the following day. The obtained sequences were fully consistent with the expected sequences, thus generating the humanized CD26-CD3 BiTE bispecific antibody expression plasmids.

Mammalian cell lines stably expressing high levels of the CD26-CD3 humanized bispecific BiTE antibodies were inoculated into Dynamis medium and cultured in a fed-batch mode at 37 °C, 8% CO₂, and 130 rpm. The culture supernatants were harvested and subjected to low-temperature centrifugation at 12,000 rpm for 15 minutes, and the supernatants were collected, followed by filtration through a 0.45 μm membrane to obtain the clarified culture supernatants. The clarified supernatants were subjected to chromatographic purification. The resulting products all exhibited an approximate molecular weight of 55 kDa.

The humanized bispecific BiTE antibody 19G300, derived from mouse antibody No. 79, was prepared using a method similar to that described above, and its amino acid sequence is set forth in SEQ ID NO: 58.

### Example 8. Dot Blot Assay of Purified Humanized Bispecific Antibody

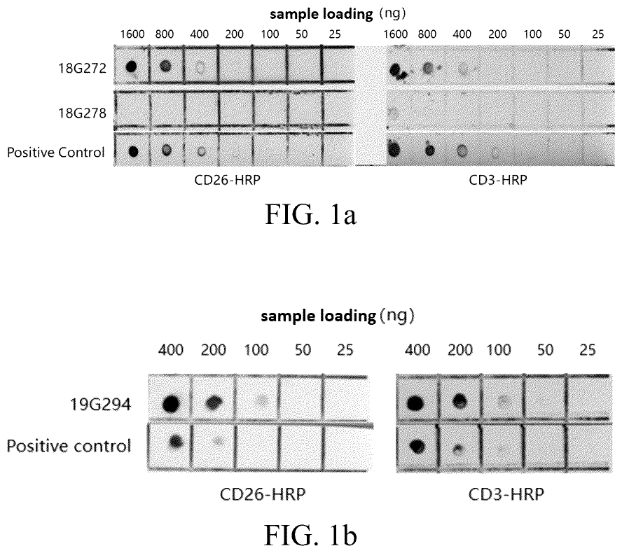

The purified antibody samples were diluted to a series of concentrations, and 2 μL of each diluted sample was spotted onto NC membranes. The positive control was 17G29. After complete absorption, the membranes were blocked with 5% skim milk blocking solution at room temperature on a horizontal shaker for 1 hour and washed three times with TBST, 5 minutes per wash. CD3-HRP protein was added at a 1:1000 dilution to 2.5% skim milk blocking solution (50 μL CD3-HRP added to 50 mL of 2.5% skim milk blocking solution), incubated at room temperature on a horizontal shaker for 1 hour, followed by three washes with TBST, 5 minutes per wash. CD26-HRP protein was added at a 1:1000 dilution to 2.5% skim milk blocking solution (50 μL CD26-HRP added to 50 mL of 2.5% skim milk blocking solution), incubated at room temperature on a horizontal shaker for 1 hour, followed by three washes with TBST, 5 minutes per wash. The TBST solution on the membrane was blotted, and the color development solution (SuperSignal West Pico Chemiluminescent Substrate) was added, allowing color development for 1 minute. The color development solution was blotted, and the membrane was exposed in a gel imaging system using a continuous exposure program set to 15 seconds per frame for 12 consecutive frames. Dot blot assays were performed on 18G272 (humanized antibody derived from mouse antibody No. 62) and 18G278 (humanized antibody derived from mouse antibody No. 212). 18G272 exhibited binding activity to both CD26 and CD3 at different concentrations comparable to the positive control 17629 (YS110-derived humanized anti-CD26-CD3 bispecific antibody) and significantly higher than 18G278 (FIG. 1-a). Dot blot assays were performed for six samples, 19G292-19G297 (humanized anti-CD26-CD3 bispecific antibodies derived from mouse antibody No. 62 and No. 212). All six samples demonstrated binding activity to both CD26 and CD3. The binding activity of 19G292-19G296 to CD26 was comparable to the positive control 17G29, and 19G294 even surpassed the positive control (FIG. 1-b).

### Example 9. Study on the Binding Specificity of Humanized Bispecific Antibody to Target Cell Antigen

### 1. Screening of CD26-Positive and CD26-Negative Cells

Target cells were cultured in T75 cell culture flasks until reaching over 80% confluence. Cells were then digested with trypsin, collected, and washed once with PBS. Cell counting was performed using a hemocytometer, and the cells were aliquoted into portions containing 5×10⁵ cells each. A monoclonal anti-CD26 antibody was used as the primary antibody and incubated with the target cells at room temperature for approximately 40 minutes. After incubation, the cells were centrifuged, and the supernatant was discarded. The cell pellet was resuspended in PBS, centrifuged again, and the supernatant was discarded. The cell pellet was then collected. Subsequently, an Alexa Fluor 488-conjugated mouse anti-human IgG1 antibody was used as the secondary antibody. The cell pellet was resuspended and incubated at room temperature for approximately 30 minutes in the dark. After incubation, the cells were washed twice with PBS, centrifuged and the supernatant was discarded, and the cell pellet was collected. The cell pellet was resuspended in approximately 200 μL of PBS solution and flow cytometry was performed within 1 hour to determine CD26 positivity rate.

The expression of CD26 was determined by flow cytometry. The cell lines 786-0, OS-RC-2, A498, NCI-H226, NCI-H2052, NCI-H596, HCC827, Huh-7, and PC-3 were identified as CD26-positive, while G401 and A-375 were identified as CD26-negative. The CD26-positive rate of each cell line is shown in Table 4. The CD26-positive cell lines are consistent with those reported in the literature (Chinese Patent Application No. CN200680034937.4; CD26/DPP4-a potential biomarker and target for cancer therapy, Pharmacology & Therapeutics (2019), 198:135-159).

TABLE 4
CD26 Positivity Rate of Target Cells

| | | | CD26 positivity |
|---|---|---|---|
| No. | Cell line name | Tumor type | rate |
| 1 | 786-0 | Kidney cancer | 99.9% |
| 2 | OS-RC-2 | | 97.8% |
| 3 | A498 | | 97.1% |
| 4 | G401 | | 0% |
| 5 | NCI-H226 | Mesothelioma | 99.4% |
| 6 | NCI-H2052 | | 88.2% |
| 7 | NCI-H596 | Lung cancer | 72.1% |
| 8 | HCC 827 | | 93.8% |
| 9 | Huh-7 | Liver cancer | 95.3% |
| 10 | PC-3 | Prostate cancer | 93.8% |
| 11 | A-375 | Melanoma | 2.90% |

### 2. FITC Labeling of Antibody

FITC was weighed and dissolved in DMSO to a final concentration of 1 mg/mL. Separately, 500 μL of the antibody 18G272 was adjusted to a final concentration of 1 mg/mL, followed by adding 1/10 volume of 1 M Na₂CO₃ solution with thorough mixing. Subsequently, 15 μL of the FITC solution was added, mixed well, and the mixture was incubated at room temperature in the dark for 3.5 hours. After incubation, the mixture was centrifuged using an ultrafiltration tube with an interception diameter of 10KDa to completely remove the FITC that was not bound to 18G272, and the solution in the tube was collected.

### 3. Flow Cytometry Analysis

Cells cultured in T75 cell culture flasks to approximately 80% confluence were digested with trypsin and collected. The cells were resuspended in an appropriate volume of PBS, counted using a hemocytometer, and aliquoted into portions containing 5×10⁵ cells each. The cells were centrifuged to collect the pellet, which was then resuspended with FITC-labeled antibody 18G272 and incubated on a tube rotator at room temperature in the dark for 30 minutes. After incubation, the cells were resuspended in PBS, centrifuged, and the supernatant was discarded, and the procedure was repeated once. The cell pellet was resuspended in 500 μL PBS. Negative control: In another sample, no antibody sample was added in the labeling process, the remaining steps were performed in parallel with the above process, and the resulting solution served as the FITC-labeled negative control. A BD Accuri C6 flow cytometer was used to determine the positive rate of FITC labeling.

The results demonstrated that the humanized bispecific antibody 18G272 bound to CD26-positive 786-0, NCI-H226, PC-3, and HCC827 cells, but did not bind to CD26-negative A375 cells, indicating its specificity for binding to the CD26 target.

The same method was used to evaluate the binding specificity of the humanized bispecific antibody 19G294 to target cell antigens. 19G294 bound to CD26-positive 786-0, NCI-H226, PC-3, and OS-RC-2 cells, but did not bind to CD26-negative G401 cells. The humanized bispecific antibody 19G294 binds to CD26-positive cells but does not bind to CD26-negative cells, demonstrating its binding to the CD26 antigen was specific.

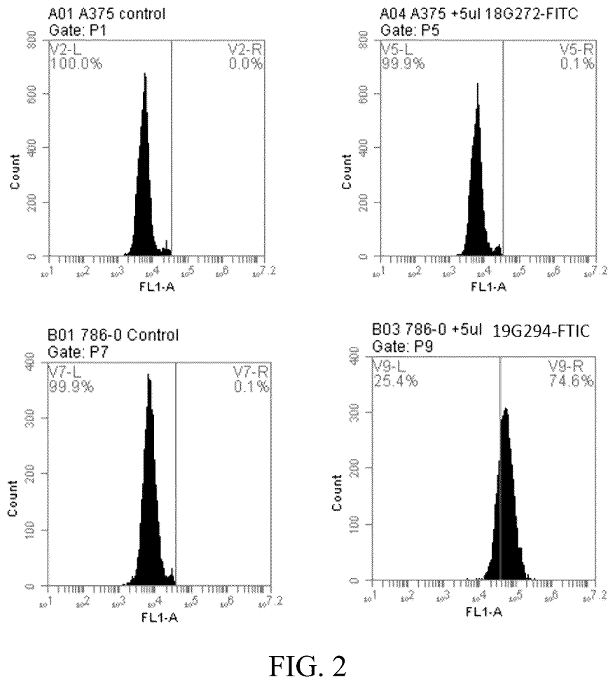

Representative flow cytometry plots illustrating antibody binding specificity to target cell antigens are shown in FIG. 2, with detailed data provided in Tables 5 and 6.

TABLE 5
Binding specificity of antibody 18G272 to target cell
antigens as determined by flow cytometry

| cell name | 786-0 | NCI-H226 | PC-3 | HCC 827 | A375 |
|---|---|---|---|---|---|
| specific binding | 74.6% | 80.7% | 64.9% | 41.7% | 0.1% |
| rate | | | | | |

TABLE 6
Binding specificity of antibody 19G294 to target cell antigens
as determined by flow cytometry

| cell name | 786-0 | NCI-H226 | NCI-H2052 | PC-3 | OS-RC-2 | G401 |
|---|---|---|---|---|---|---|
| specific | 79.0% | 79.5% | 70.4% | 50.6% | 91.6% | 0.0% |
| binding | | | | | | |
| rate | | | | | | |

Other humanized bispecific antibodies disclosed herein also bound specifically to CD26-positive cells and did not bind to CD26-negative cells, demonstrating specific binding to the CD26 antigen target.

### Example 10. Affinity Analysis of Humanized Bispecific Antibody for CD26 Protein

### 1. Affinity Analysis of Antibody for CD26 Protein

CD26 protein was first biotinylated using EZ-Link NHS-PEG12-Biotin, and the biotinylated protein was immobilized on SA biosensors. The biosensors were then equilibrated in 1× Kinetics solution, followed by incubation with the test antibody solution to allow binding. The concentrations of the antibody to be tested were 31.3 nM, 62.5 nM, 125 nM, 250 nM, and 500 nM. Afterward, the biosensors were placed in 1× Kinetics solution for dissociation. Data were analyzed using ForteBio Data Analysis 8.0 software to calculate the affinity constant values.

TABLE 7
Affinity of humanized bispecific antibody for CD26 Protein

| Sample | KD | kon | kdis |
|---|---|---|---|
| name | (M) | (1/Ms) | (1/s) |
| 19G294 | 0.914E−09 | 9.24E+04 | 8.45E−05 |
| 19G292 | <1.0E−12 | 6.58E+04 | No dissociation |
| 19G293 | <1.0E−12 | 5.46E+04 | No dissociation |
| 19G296 | 1.48E−08 | 9.45E+04 | 1.40E−03 |
| 19G297 | 4.40E−09 | 9.18E+04 | 3.70E−04 |
| 17G29 | 1.18E−09 | 1.83E+05 | 2.16E−04 |
| 21G587 | 0.352E−09 | 1.14E+05 | 4.01E−05 |

The above data indicate that, when the anti-CD3 segment is the same, humanized antibodies derived from mouse antibody No. 62 (19G292, 19G293, 19G294) exhibit lower affinity constant values than those derived from mouse antibody 212 or YS110, indicating that the former have a higher affinity for CD26. Additionally, 19G294 and 21G587 have the same anti-CD26 segment but have different anti-CD3 segments; both exhibit comparable affinity toward CD26 protein.

Based on the affinity data (Table 7), when the anti-CD3 segment is the same, humanized bispecific antibodies derived from mouse antibody No. 62 (19G292, 19G293, 19G294) demonstrate superior binding ability to CD26 protein compared with those derived from mouse antibody 212 (19G296, 19G297) or YS110 (17G29). When the anti-CD3 segment differs, humanized bispecific antibodies derived from mouse antibody No. 62 (19G294, 21G587) show equivalent binding ability to CD26 protein. These results indicate that the antibodies of the present disclosure recognize and bind to CD26 protein without being affected by the fusion of different anti-CD3 segments or their binding to CD3 protein.

### 2. Simultaneous Binding of Bispecific Antibody to CD26 and CD3 Proteins

The 19G294 protein was biotinylated using EZ-Link NHS-PEG12-Biotin, and the biotinylated 19G294 protein was immobilized on SA biosensors. The biosensors were equilibrated in 1× Kinetics solution. The immobilized 19G294 protein was first exposed to 400 nM CD26 protein solution for binding, followed by immersion in CD26 solution (400 nM) containing 400 nM CD3 protein. Or, the immobilized 19G294 protein was first exposed to 400 nM CD3 protein solution for binding, followed by immersion in CD3 solution (400 nM) containing 400 nM CD26 protein. Binding interactions were monitored using ForteBio Data Analysis 8.0 software.

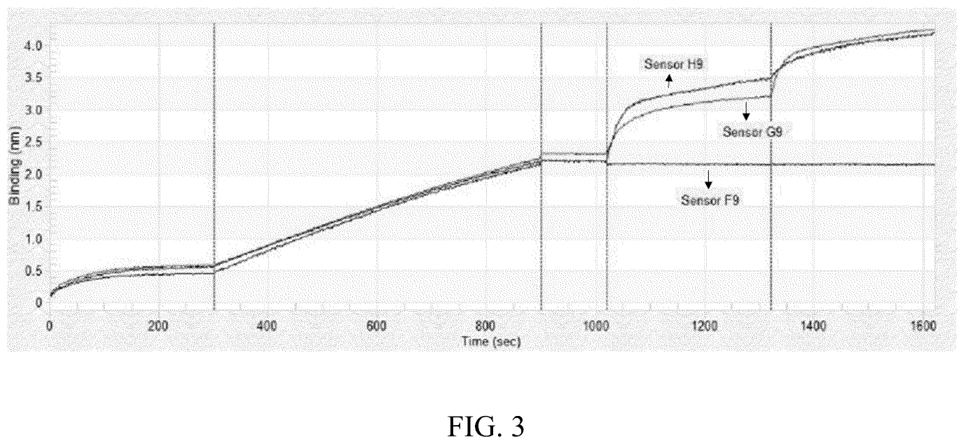

As shown in FIG. 3, when 19G294 was immobilized on SA biosensors and bound to CD26 protein to saturation, it was still able to bind CD3 protein subsequently (FIG. 3, Sensor G9). Conversely, when bound to CD3 protein to saturation, 19G294 was still able to bind CD26 protein subsequently (FIG. 3, Sensor H9). Sensor F9 in FIG. 3 served as a negative control, demonstrating that no nonspecific binding of the sensor to the solution occurred. The results indicate that, 19G294 can still bind to CD3 protein after first binding to CD26 protein, and can still bind to CD26 protein after first binding to CD3 protein, demonstrating that there is no preferred order of binding. Binding to CD3 protein does not affect the recognition and binding of the humanized bispecific antibody to CD26.

### Example 11. Evaluation of Humanized Bispecific Antibody-Mediated PBMC Cytotoxicity Against Target Cells

### 1. 786-0 Cell (Human Renal Cancer Cell) Model

In the 786-0 cell reaction system, 786-0 cells were labeled with a green fluorescent signal using fluorescent dye Calcein-AM. The 786-0 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/mL, 10 ng/mL, 1 ng/mL, 0.1 ng/mL, 0.01 ng/mL, and 0.001 ng/mL, respectively. To the blank control wells, 50 μL of culture medium was added; to the positive control wells, 50 μL of 1% Triton X-100 (final concentration) was added. Then 50 μL of PBMCs at a concentration of 6×106 cells/ml were added at an ET ratio of 10:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 2. OS-RC-2 Cell (Human Renal Cell Cancer) Model

In the OS-RC-2 cell reaction system, OS-RC-2 cells were labeled with a green fluorescent signal using fluorescent dye Calcein-AM. The OS-RC-2 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/ml, 10 ng/mL, 1 ng/mL, 0.1 ng/mL, 0.01 ng/mL, and 0.001 ng/ml, respectively. To the blank control wells, 50 μL of culture medium was added; to the positive control wells, 50 μL of 1% Triton X-100 (final concentration) was added. Then 50 μL of PBMCs at a concentration of 9×106 cells/ml were added at an E/T ratio of 15:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 3. NCI-H226 Cell (Human Mesothelioma) Model

In the NCI-H226 cell reaction system, NCI-H226 cells were labeled with a green fluorescent signal using fluorescent dye Calcein-AM. The NCI-H226 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/mL, 10 ng/ml, 1 ng/mL, 0.1 ng/ml, 0.01 ng/mL, and 0.001 ng/ml, respectively. To the blank control wells, 50 μL of culture medium was added; to the positive control wells, 50 μL of 1% Triton X-100 (final concentration) was added. Then 50 μL of PBMCs at a concentration of 9×106 cells/ml were added at an E:T ratio of 15:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 4. NCI-H2052 Cell (Human Mesothelioma) Model

In the NCI-H2052 cell reaction system, NCI-H2052 cells were labeled with a green fluorescent signal using fluorescent dye Calcein-AM. The NCI-H2052 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/mL, 10 ng/mL, 1 ng/mL, 0.1 ng/mL, 0.01 ng/mL, and 0.001 ng/mL, respectively. To the blank control wells, 50 μL of culture medium was added; to the positive control wells, 50 μL of 1% Triton X-100 (final concentration) was added. Then 50 μL of PBMCs at a concentration of 9×106 cells/ml were added at an E:T ratio of 15:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 5. PC-3 Cell (Human Prostate Cancer) Model

In the PC-3 cell reaction system, PC-3 cells were labeled with a green fluorescent signal using fluorescent dye Calcein-AM. The PC-3 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/mL, 10 ng/mL, 1 ng/mL, 0.1 ng/mL, 0.01 ng/mL, and 0.001 ng/mL, respectively. To the blank control wells, 50 μL of culture medium was added; to the positive control wells, 50 μL of 1% Triton X-100 (final concentration) was added. Then 50 μL of PBMCs at a concentration of 9×106 cells/ml were added at an E:T ratio of 15:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 6. HCC 827 Cell (Human Non-Small Cell Lung Cancer) Model

In the HCC 827 cell reaction system, HCC 827 cells were labeled with a green fluorescent signal using fluorescent dye Calcein-AM. The HCC 827 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/mL, 10 ng/mL, 1 ng/mL, 0.1 ng/mL, 0.01 ng/mL, and 0.001 ng/mL, respectively. To the blank control wells, 50 μL of culture medium was added; to the positive control wells, 50 μL of 1% Triton X-100 (final concentration) was added. Then 50 μL of PBMCs at a concentration of 6×106 cells/ml were added at an E:T ratio of 10:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 7. Experimental Results

Cell lysis rate (%)=[(Vsample−Vvehicle control)/(VTriton X-100−Vvehicle control)]×100%

(Vsample is the mean value of the fluorescence signal reading of the drug-treated group. Vvehicle control is the mean value of the fluorescence signal reading of the blank control group. VTriton X-100 is the mean value of the fluorescence signal reading of the positive control group.)

The IC50 values of different antibody-mediated PBMC cytotoxicity against different target cells were calculated using GraphPad Prism 7.00 software based on the cell lysis rates and sample concentrations.

TABLE 9
IC50 values of different humanized bispecific antibody-mediated PBMC cytotoxicity against different target cells (pg/ml)

| cell name | 18G272 | 19G292 | 19G293 | 19G294 | 19G295 | 19G296 | 19G297 | 17G29 | YS110 |
|---|---|---|---|---|---|---|---|---|---|
| 786-0 | 2275 | 805.5 | 974 | 260.0 | Inactive | Inactive | Inactive | 1034 | >100 ng/ml |
| OS-RC-2 | 283.7 | 1043 | 2246 | 544.9 | Inactive | Inactive | Inactive | 958.1 | >100 ng/ml |
| NCI-H226 | 142.8 | 1102 | 404.6 | 752.3 | Inactive | Inactive | Inactive | 917.6 | >100 ng/ml |
| PC-3 | 378.8 | 339.2 | 564.9 | 219.6 | Inactive | Inactive | Inactive | 1140 | >100 ng/ml |
| HCC 827 | - | - | - | 546.4 | - | - | - | - | - |
| NCI-H2052 | - | - | - | 5053 | - | - | - | - | - |

-: means not measured

Humanized anti-CD26-CD3 bispecific antibodies derived from mouse antibody No. 62 (e.g., 18G272, 19G292, 19G293, 19G294) were all able to mediate PBMC cytotoxicity against various CD26-positive tumor cells exhibiting cytotoxicity activity in vitro. In contrast, humanized anti-CD26-CD3 bispecific antibodies derived from mouse antibody No. 212 (e.g., 19G295, 19G296, 19G297) were all unable to mediate PBMC cytotoxicity against CD26-positive tumor cells.

These results indicate that, mouse antibody No. 62 has superior cytotoxicity activity against CD26-positive tumor cells. The humanized anti-CD26-CD3 antibodies derived from mouse antibody No. 62 exhibit superior cytotoxicity against various CD26-positive tumors compared with 17G29 (a humanized anti-CD26-CD3 antibody derived from YS110 with the same CD3 antibody segment) and the humanized anti-CD26-CD3 antibodies derived from mouse antibody No. 212 with the same CD3 antibody segment. Additionally, the mouse antibody No. 62, after humanization, can be used in BiTE-format bispecific antibodies (18G272, 19G292, 19G293, 19G294, 17G29), and the cytotoxicity against various CD26-positive tumor cells are superior to the full-length IgG antibody YS110.

### Example 12. Activation of PBMCs by the Humanized Bispecific Antibody During the Mediation of PBMC Cytotoxicity Against Target Cells

PBMC+bispecific antibody system: PBMCs were collected, counted, and resuspended to 1×106 cells/mL, 200 μL per well was added to 12-well cell culture plates. Antibody was diluted to a final concentration of 1 ng/mL. Co-incubated for 24 hours.

PBMC+bispecific antibody+OS-RC-2 cell system: PBMCs were collected, counted, and resuspended at 6×105 cells/mL, 100 μL per well was added to 12-well cell culture plates. Antibody was diluted to a final concentration of 1 ng/mL. OS-RC-2 cells were diluted to 9×106 cells/mL, and 100 μL per well was added to the corresponding wells of the 12-well plates. Co-incubated for 24 hours.

After incubation, centrifugation was performed and the cell pellet was collected and resuspended in PBS. Anti-CD25-APC antibody (purchased from Miltenyi) and anti-CD69-PE antibody (purchased from Miltenyi) were added, incubated at 4 °C for 10 minutes. After the incubation, the cells were washed twice with PBS, resuspended in PBS and then analyzed using a BD Accuri C6 flow cytometer.

In the “PBMC+bispecific antibody” system, the bispecific antibody did not induce upregulation of CD25 and CD69 expression on PBMCs. In the “PBMC+bispecific antibody+OS-RC-2 cell” system, the bispecific antibody induced upregulation of CD25 and CD69 expression on PBMCs, thereby promoting the activation of PBMCs. In the “PBMC+YS110+OS-RC-2 cell” system, YS110 did not induce upregulation of CD25 and CD69 expression on PBMCs. See Table 10.

TABLE 10
Expression of CD25 and CD69 on PBMCs during antibody-mediated
PBMC cytotoxicity against target cells as determined by flow cytometry.”

| | | | | PBMC + | PBMC + | PBMC + |
|---|---|---|---|---|---|---|
| reaction | | | | 18G272 + | 19G294 + | YS110 + |
| system | PBMC | PBMC + 18G272 | PBMC + 19G294 | OS-RC-2 | OS-RC-2 | OS-RC-2 |
| CD25 | 2.4% | 5.0% | 3.5% | 23.7% | 23.5% | 2.1% |
| expression | | | | | | |
| level | | | | | | |
| CD69 | 5.2% | 10.3% | 9.8% | 55.5% | 58.9% | 4.7% |
| expression | | | | | | |
| level | | | | | | |

The same phenomenon was also observed in other CD26-positive tumor cells such as 786-0. In the “PBMC+bispecific antibody” system, the bispecific antibody did not induce upregulation of CD25 and CD69 expression on PBMCs; whereas in the “PBMC+bispecific antibody+other CD26-positive tumor (786-0, etc.) cells” system, the bispecific antibody induced upregulation of CD25 and CD69 expression on PBMCs.

CD25 and CD69 are important markers of T cell activation. The above results demonstrate that the full-length IgG antibody YS110 cannot effectively induce T cell activation, and that the CD26-CD3 bispecific antibody does not induce significant T cell activation prior to reaching the tumor site. T cell activation occurs only after the antibody binds to tumor cells, which leads to activation of T cells in the vicinity of the tumor tissue and subsequent tumor cell killing, thereby enhancing therapeutic safety.

### Example 13. Cytokine Detection During Humanized Bispecific Antibody-Mediated PBMC Cytotoxicity Against Target Cells

OS-RC-2 cells were resuspended at 6×105 cells/mL, and 100 μL per well was added to 12-well plates. PBMCs from individual donors were collected and resuspended at 9×106 cells/mL, and 100 μL per well was added. Then, 100 μL of each sample (19G294, 18G272, 17G29) with concentrations of 0 ng/ml, 3 ng/mL was added to the corresponding wells, resulting in a final volume of 300 μL per well. The reaction system was incubated at 37 °C for 2, 4, 6, and 21 hours. After incubation, the culture supernatants were harvested, and subjected to centrifugation. Then the supernatants were taken for ELISA to detect cytokine IFN-γ, TNF-α, IL-4, and IL-10 using ELISA kits.

The cytokine levels detected in each sample using the ELISA kits are shown in the table below.

TABLE 11
IFN-γ level detected by ELISA (pg/ml)

| | 2 h | 4 h | 6 h | 21 h |
|---|---|---|---|---|
| 0 ng/ml | −7.65 | 143.7 | −58.3 | 37.7 |
| 18G272 | 116.7 | 667.7 | 964.7 | 1181.7 |
| 19G294 | 91.7 | 337.7 | 303.7 | 398.7 |
| 17G29 | 341.7 | 1395.7 | 1777.7 | 2368.7 |

TABLE 12
TNF-α level detected by ELISA (pg/ml)

| | 2 h | 4 h | 6 h | 21 h |
|---|---|---|---|---|
| 0 ng/ml | 16.01 | 19.55 | 17.56 | 10.33 |
| 18G272 | 161.3 | OVERFLOW | 192.51 | 158.73 |
| | | * | | |
| 19G294 | 111.88 | 166.88 | 111.79 | 47.22 |
| 17G29 | 165.43 | 182.13 | 183.73 | 180.33 |

TABLE 13
IL-10 level detected by ELISA (pg/ml)

| | 2 h | 4 h | 6 h | 8 h |
|---|---|---|---|---|
| 0 ng/ml | 8.4 | 16.5 | 19.3 | 28.9 |
| 18G272 | 15.9 | 53.7 | 86.2 | 190.6 |
| 19G294 | 9.9 | 32.3 | 47.3 | 65.9 |
| 17G29 | 22.8 | 113.1 | 90.2 | 200.4 |

TABLE 14
IL-4 level detected by ELISA (pg/ml)

| | 2 h | 4 h | 6 h | 21 h |
|---|---|---|---|---|
| 0 ng/ml | 3.56 | 4.08 | 3.44 | 3.05 |
| 18G272 | 7.67 | 19.21 | 21.64 | 6.64 |
| 19G294 | 3.69 | 10.74 | 11.38 | 3.69 |
| 17G29 | 23.82 | 58.69 | 57.79 | 43,44 |

* Indicates a value exceeding
the device's detection limit

In summary, during the humanized bispecific antibody-mediated PBMC cytotoxicity against target cells, under the same dosing conditions, the cytokine levels secreted by PBMCs induced by anti-CD26-CD3 humanized bispecific antibodies 19G294 and 18G272 (derived from mouse antibody No. 62) were lower at all time points compared with 17G29 (an anti-CD26-CD3 humanized bispecific antibody derived from YS110). For this type of CD3-targeting and T cell-activating molecular structure, low cytokine storm caused by T cell-secreted cytokines is associated with improved safety. (A FDA oncology analysis of CD3 bispecific constructs and first-in-human dose selection (2017), Regulatory Toxicology and Pharmacology, 90:144-152). Under the premise of the same CD3-recognizing region and equivalent experimental conditions, compared with 17G29, the bispecific antibodies 19G294 and 18G272 of this disclosure demonstrate the advantage of inducing lower levels of T cell-secreted cytokines. This may be related to the fact that the CD26 antigen fragment recognized by their CDR regions is different from that recognized by 17G29. Compared with other CD26 antigen fragments, the fragment recognized by the antibodies of the present disclosure is associated with reduced cytokine storm risk and improved safety.

### Example 14: Humanized Bispecific Antibody-Mediated PBMC Cytotoxicity Against PBMCs

### 1. 786-0 Cell Model

PBMCs were labeled with a green fluorescent signal using fluorescent dye Calcein-AM. 786-0 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/ml, 10 ng/mL, 1 ng/mL, 0.1 ng/mL, 0.01 ng/mL, and 0.001 ng/mL, respectively. To the blank control wells, 50 μL of culture medium was added; to the positive control wells, 50 μL of 1% Triton X-100 (final concentration) was added. Then 50 μL of PBMCs at a concentration of 9×106 cells/mL were added at an E:T ratio of 10:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 2. OS-RC-2 Cell Model

PBMCs were labeled with a green fluorescent signal using fluorescent dye Calcein-AM. OS-RC-2 cells were seeded into a U-bottom 96-well cell culture plate at a cell concentration of 6×105 cells/ml (50 μL per well). To the corresponding sample reaction wells, 50 μL of humanized bispecific antibody was added at final concentrations of 100 ng/ml, 10 ng/mL, 1 ng/mL, 0.1 ng/mL, 0.01 ng/mL, and 0.001 ng/mL, respectively. To the blank control wells, 50 μL of culture medium was added; to the positive control wells, 50 μL of 1% Triton X-100 (final concentration) was added. Then 50 μL of PBMCs at a concentration of 9×106 cells/mL were added at an E:T ratio of 15:1. The reaction system was incubated at 37 °C under CO2 for 5 hours. After completion of the reaction, centrifugation was performed, and the supernatants were collected and transferred to a new 96-well plate. After a second centrifugation, 80 μL of the supernatant from each well was collected and transferred to a black 96-well plate, and fluorescence intensity was measured using a microplate reader at 470 nm excitation wavelength and 515 nm emission wavelength.

### 3. Results and Analysis

Cell lysis rate (%)=[(Vsample−Vvehicle control)/(VTriton X-100−Vvehicle control)]×100%

(Vsample is the mean value of the fluorescence signal reading of the drug-treated group. Vvehicle control is the mean value of the fluorescence signal reading of the blank control group. VTriton X-100 is the mean value of the fluorescence signal reading of the positive control group.)

The IC50 values of PBMC cytotoxicity against PBMCs mediated by each antibody sample was calculated using GraphPad Prism 7.00 software based on the cell lysis rates and sample concentrations.

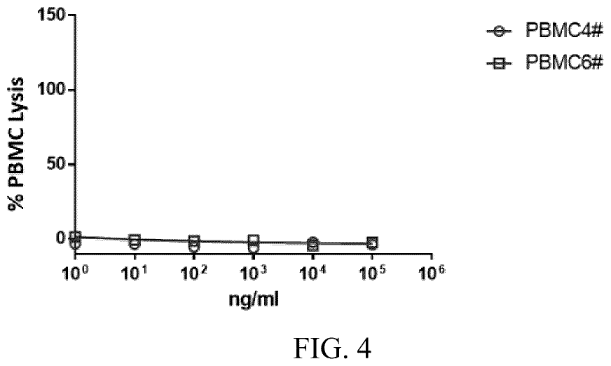

During the antibody 18G272-mediated PBMC cytotoxicity against 786-0 target cells, antibody-mediated PBMC cytotoxicity against PBMCs was extremely low. The IC₅₀ of the cytotoxic effect of PBMC (#4) against PBMC (#4) mediated by antibody 18G272 was approximately 99,730,000 pg/mL; and the antibody 18G272 did not mediate PBMC (#6) cytotoxic effect against PBMC (#6). The results are shown in FIG. 4.

During the antibody 18G272-mediated PBMC cytotoxicity against OS-RC-2 target cells, the antibody 18G272 did not mediate PBMC cytotoxicity against PBMCs. The antibody 18G272 did not mediate PBMC (#1) cytotoxic effect against PBMC (#1), and the antibody 18G272 did not mediate PBMC (#3) cytotoxic effect against PBMC (#3).

During the antibody 19G294-mediated PMBC cytotoxicity against 786-0 target cells, antibody-mediated PBMC cytotoxicity against PBMCs was extremely low. The IC₅₀ of the cytotoxic effect of PBMC (#2) against PBMC (#2) mediated by antibody 19G294 was approximately 1,915,313 pg/mL; and the IC₅₀ of the cytotoxic effect of PBMC (#3) against PBMC (#3) mediated by antibody 19G294 was approximately 1,947,224 pg/mL.

During the antibody 19G294-mediated PMBC cytotoxicity against OS-RC-2 target cells, antibody-mediated PBMC cytotoxicity against PBMCs was extremely low. The IC₅₀ of the cytotoxic effect of PBMC (#2) against PBMC (#2) mediated by antibody 19G294 was approximately 47,920,315 pg/mL; and the IC₅₀ of the cytotoxic effect of PBMC (#3) against PBMC (#3) mediated by antibody 19G294 was approximately 1,406,115,138 pg/mL.

In summary, the CD26-CD3 humanized bispecific antibodies can accurately recognize PBMCs. Although CD26 expression on T cells has been reported in the literature, the bispecific antibodies 18G272 and 19G294 of the present disclosure do not mediate significant PBMC cytotoxicity against PBMCs, demonstrating their favorable safety profile.

### Example 15: In Vitro Efficacy Comparison of 19G294 and BAVENCIO (Anti-PD-L1 Antibody)

Target cells were cultured in T75 cell culture flasks until reaching over 80% confluence. Cells were then digested with trypsin, collected, and washed once with PBS. Cell counting was performed using a hemocytometer, and the cells were aliquoted into portions containing 5×10⁵ cells each. Target cells were incubated with PE-labeled anti-PD-L1 monoclonal antibody (purchased from Sino Biological) at room temperature in the dark for approximately 40 min. After incubation, the cells were centrifuged, and the supernatant was discarded. The cell pellet was resuspended in PBS, centrifuged again, and the supernatant was discarded. The cell pellet was then collected. The cell pellet was resuspended in approximately 200 μL of PBS solution, and flow cytometry was performed within 1 hour to determine the PD-L1 positivity rate.

Flow cytometry analysis determined that the PD-L1 positivity rate of 786-0 cell line was 66.2%, and the PD-L1 positivity rate of A498 was 29.6%.

TABLE 15
Analysis of PD-L1 positivity rate in cells

| | 786-0 | A498 |
|---|---|---|
| PD-L1 | 66.2% | 29.6% |

Human renal cancer 786-0-LUC cells (786-0 cells transfected with the luciferase reporter gene) and human renal cancer A498-LUC cells (A498 cells transfected with the luciferase reporter gene) were seeded into white opaque 96-well cell culture plates at a cell concentration of 3×105 cells/mL (50 μL per well). In the corresponding reaction wells, 50 μL of antibody was added at final concentrations of 100 ng/ml, 10 ng/mL, 1 ng/mL, 0.1 ng/mL, 0.01 ng/mL, and 0.001 ng/mL, respectively. Then 50 μL of PBMCs at a concentration of 3×106 cells/mL were added at an E:T ratio of 10:1. The reaction system was incubated at 37 °C under CO2 for 21 hours. After completion of the reaction, centrifugation was performed, and the supernatants were discarded, and 100 μL of reporter gene assay reagent (purchased from Vazyme) was added. Plates were shaken at 400 rpm on a microplate shaker for 5 min, and measured using a microplate reader under luminescence (Lum) conditions immediately. The assay data were analyzed using GraphPad Prism 7.00 software. A four-parameter curve was generated based on the reduction in reporter gene and antibody dose, and the EC50 values were calculated. The results are shown in Table 16.

TABLE 16
Antibody-mediated PBMC cytotoxicity
against target cells (EC50 pg/ml)

| | 786-0-LUC | A498-LUC |
|---|---|---|
| 19G294 | 2623 | 3211 |
| BAVENCIO | 1791039 | 3289316 |

The data above indicate that the EC₅₀ values of PBMC cytotoxicity mediated by 19G294 against the two target cells were significantly lower than those of BAVENCIO. The PD-L1 expression rates of 786-0 and A498 cells were 66.2% and 29.6%, respectively. These results demonstrate that 19G294 exhibits significantly higher activity than BAVENCIO regardless of the PD-L1 expression level of the target cells.

### Example 16. In Vitro Efficacy Comparison Between 21G587 and 19G294

Human renal cancer 786-O-LUC cells (786-0 cells transfected with the luciferase reporter gene) and human renal cancer A498-LUC cells (A498 cells transfected with the luciferase reporter gene) were seeded into white opaque 96-well cell culture plates at a cell concentration of 3×105 cells/mL (50 μL per well). In the corresponding reaction wells, 50 μL of antibody was added at final concentrations of 100 ng/ml, 10 ng/mL, 1 ng/mL, 0.1 ng/mL, 0.01 ng/mL, and 0.001 ng/mL, respectively. Then 50 μL of PBMCs at a concentration of 3×106 cells/mL were added at an ET ratio of 10:1. The reaction system was incubated at 37 °C under CO2 for 21 hours. After completion of the reaction, centrifugation was performed, and the supernatants were discarded, and 100 μL of reporter gene assay reagent (purchased from Vazyme) was added. Plates were shaken at 400 rpm on a microplate shaker for 5 min, and measured using a microplate reader under luminescence (Lum) conditions immediately. The assay data were analyzed using GraphPad Prism 7.00 software. A four-parameter curve was generated based on the reduction in reporter gene and antibody dose, and the EC50 values were calculated. The results are shown in Table 17.

TABLE 17
Antibody-mediated PBMC cytotoxicity
against PBMCs (EC50 pg/ml)

| | 786-0-LUC | A498-LUC |
|---|---|---|
| 21G587 | 2493 | 5397 |
| 19G294 | 1428 | 4153 |

The data above indicate that when the anti-CD26 antibody fragment of the present disclosure, was combined with different anti-CD3 fragments, 19G294 and 21G587 had the same anti-CD26 segment and differ in the anti-CD3 segments, both demonstrated equivalent levels of in vitro efficacy. The in vitro efficacy experiments of the present study further confirmed that the recognition and binding to CD26 protein fragment of antibodies of the present disclosure are not affected by the fusion of different anti-CD3 segments and their binding to CD3 protein.

### Example 17. Efficacy of Humanized Bispecific Antibodies 18G272 and 19G294 in NOD/SCID Mice Bearing PC-3 (Prostate Cancer) Xenografts

Methods: NOD/SCID mice weighing 18-22 g and aged approximately 5-7 weeks were selected. A suspension of PC-3 (5×106 cells/0.1 mL) was thoroughly mixed with a suspension of PBMCs (1×107 cells/0.1 mL) at a 1:1 volume ratio, and 0.2 mL of the mixture was inoculated subcutaneously into each mouse. The animals were randomly divided into three groups based on body weight: Model Group (PBS), 19G294 Group (30 μg/mouse/dose), and 18G272 Group (30 μg/mouse/dose), with six animals in each group. The Model Group and treatment groups received intravenous administration starting on the day of tumor cell inoculation, with dosing for five consecutive days. After a two-day treatment-free interval, a second course of dosing was conducted. The first dose was administered 1 hour after subcutaneous inoculation of the cells. Thus, the first course was administered on Days 1, 2, 3, 4, and 5 (D1-D5), and the second course was administered on Days 8, 9, 10, 11, and 12 (D8-D12), with a dosing frequency of once daily.

General clinical observations: During the quarantine period and the study period, animals were observed at least once daily, including tumor growth and the effects of treatment on normal behavior. Specific observations included death or moribundity of the tumor-bearing animals, mental state, behavioral activity, and other abnormalities.

Body weight: Measured 2-3 times per week.

Tumor Volume: Measured 2-3 times per week using a vernier caliper to determine the length and width of the tumor.

Tumor volume (mm³)=(length×width 2)/2

Relative tumor growth inhibition TGI(%)=(1−T/C)×100%

Conventionally, T represents the relative tumor volume of the treatment group at a given time point (the ratio of the tumor volume measured at that time to the tumor volume at the time of group assignment), and C represents the relative tumor volume of the Model Group at the corresponding time point (the ratio of the tumor volume measured at that time to the tumor volume at the time of group assignment). In the experiments of the present disclosure, animals were grouped based on body weight on the day of inoculation. When calculating TGI, T and C represent the actual tumor volumes measured at each time point for the treatment group and the Model Group, respectively.

Tumor weight: After the final measurement, animals were euthanized, and tumor masses were excised, rinsed with physiological saline, blotted dry with filter paper, weighed, and photographed.

Relative tumor growth inhibition TGI(%)=(1−TTW/CTW)×100%
(TTW represents the average tumor weight of the treatment group at the end of the study. CTW represents the average tumor weight of the Model Group at the end of the study.)

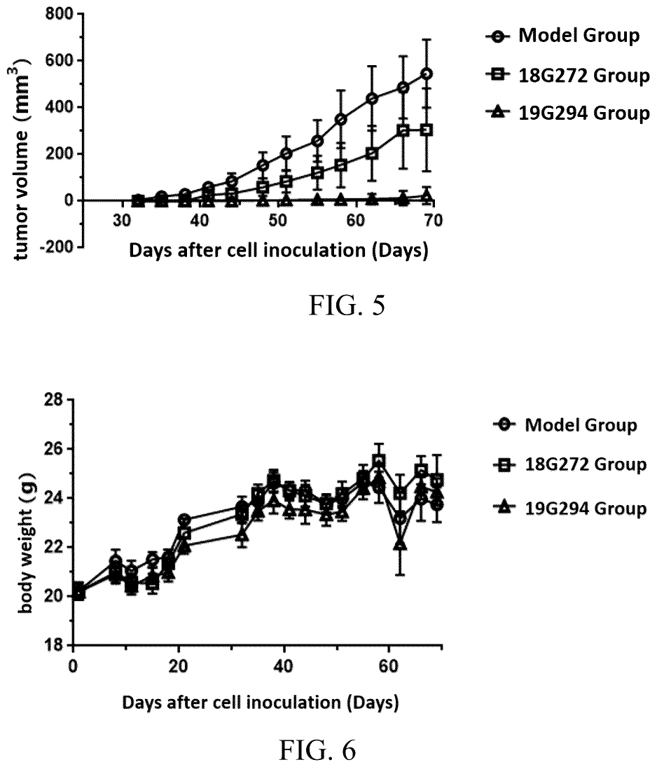

Conclusions: At a dose of 30 μg per mouse, 18G272 exhibited a tendency to inhibit tumor growth in mice bearing PC-3 (human prostate cancer) xenografts, while 19G294 almost completely inhibited tumor growth in the same model. The results are shown in FIG. 5 and Table 18. At the dose of 30 μg per mouse, animals treated with 19G294 or 18G272 showed an increasing trend in body weight, comparable to that of the Model Group, and tolerated the treatment well during the study. The results are shown in FIG. 6 and Table 18.

TABLE 18
Tumor growth and body weight in mice bearing
subcutaneous PC-3 human prostate cancer xenografts

| | D69 | D69 | D1 | D69 |
|---|---|---|---|---|
| | tumor volume | Tumor weight | body weight | body weight |
| Group | (mm3) | (mg) | (g) | (g) |
| Model | 546.05 ± 146.04 | 473.88 ± 127.65 | 20.23 ± 0.37 | 23.75 ± 0.72 |
| Group | | | | |
| 18G272 | 305.82 ± 177.85 | 225.78 ± 120.25 | 20.22 ± 0.34 | 24.77 ± 1.01 |
| Group | | | | |
| 19G294 | 23.69 ± 15.13** | 11.28 ± 7.63** | 20.20 ± 0.28 | 24.27 ± 0.56 |
| Group | | | | |

*: P < 0.05, vs. Model Group;
**: P < 0.01, vs. Model Group

### Example 18. Efficacy of Humanized Bispecific Antibody 18G272 in NOD/SCID Mice Bearing NCI-H596 (Lung Cancer) Xenografts

Methods: NOD/SCID mice weighing 18-22 g and aged approximately 5-7 weeks were selected. A suspension of NCI-H596 (8×106 cells/0.1 mL) was thoroughly mixed with a suspension of PBMCs (1.6×107 cells/0.1 mL) at a 1:1 volume ratio, and 0.2 mL of the mixture was inoculated subcutaneously into each mouse. The animals were randomly divided into two groups based on body weight: Model Group (PBS), 18G272 Group (30 μg/mouse/dose), with six animals in each group.

The administration route and dosing schedule were the same as in Example 17.

General clinical observations, body weight, and tumor volume measurements were conducted as in Example 17.

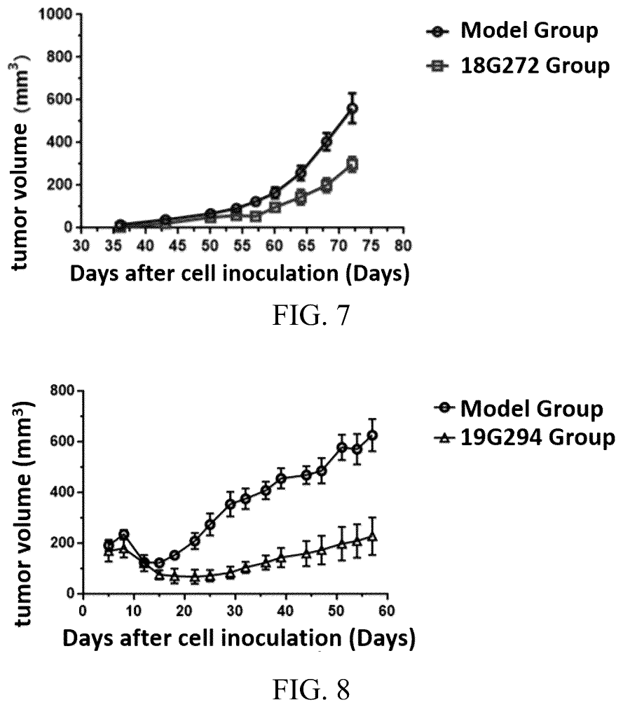

Conclusions: At a dose of 30 μg/mouse, 18G272 significantly inhibited tumor growth in mice bearing human lung adenosquamous carcinoma xenografts; at the dose of 30 μg/mouse, animals treated with 18G272 showed an increasing trend in body weight, and tolerated the treatment well during the study. The results are shown in FIG. 7 and Table 19.

TABLE 19
Tumor growth and body weight in mice bearing subcutaneous
NCI-H596 human lung adenosquamous carcinoma xenografts

| | D72 | DI | D72 |
|---|---|---|---|
| | tumor volume | body weight | body weight |
| Group | (mm3) | (g) | (g) |
| Model | 560.84 ± 70.19 | 19.22 ± 0.54 | 23.95 ± 0.74 |
| Group | | | |
| 18G272 | 298.46 ± 34.56* | 19.14 ± 0.33 | 24.52 ± 0.48 |
| Group | | | |

*: P < 0.05, vs. Model Group

### Example 19. Efficacy of Humanized Bispecific Antibody 19G294 in NOD/SCID Mice Bearing NCI-H226 (Mesothelioma) Xenografts

Methods: NOD/SCID mice weighing 18-22 g and aged approximately 5-7 weeks were selected. A suspension of NCI-H226 (5×106 cells/0.1 mL) was thoroughly mixed with a suspension of PBMCs (1.5×107 cells/0.1 mL) at a 1:1 volume ratio, and 0.2 mL of the mixture was inoculated subcutaneously into each mouse. The animals were randomly divided into two groups based on body weight: Model Group (PBS), 19G294 Group (60 μg/mouse/dose), with five animals in each group.

The administration route and dosing schedule were the same as in Example 17.

General clinical observations, body weight, and tumor volume measurements were conducted as in Example 17.

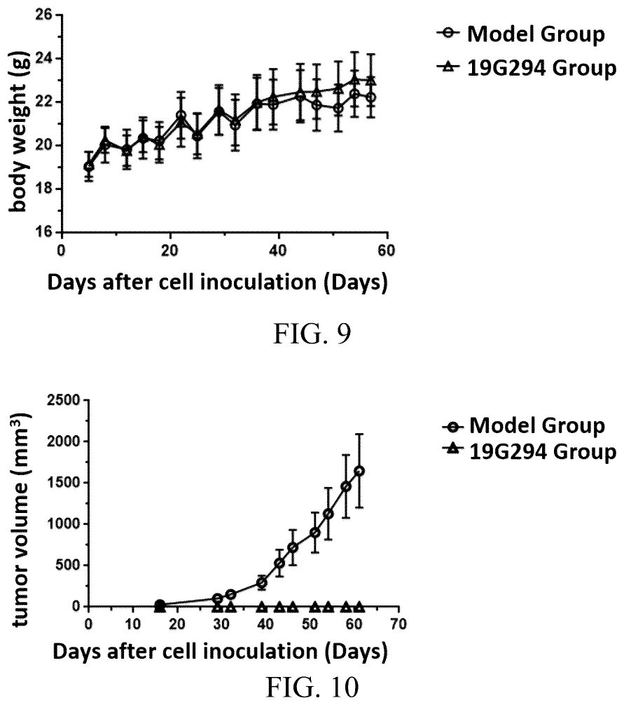

Conclusions: At a dose of 60 μg/mouse, 19G294 significantly inhibited tumor growth in mice bearing human lung adenosquamous carcinoma (Mesothelioma) xenografts, as shown in FIG. 8; at the dose of 60 μg/mouse, animals treated with 19G294 showed an increasing trend in body weight, and tolerated the treatment well during the study. The results are shown in FIG. 9 and Table 20.

TALBE 20
Tumor growth and body weight in mice bearing subcutaneous
NCI-H226 human lung adenosquamous carcinoma (Mesothelioma) xenografts

| | D57 | D57 | | DI | D57 |
|---|---|---|---|---|---|
| | tumor volume | Tumor weigh | TGI a | body weight | body weight |
| Group | (mm3) | (mg) | (%) | (g) | (g) |
| Model Group | 653.13 ± 4.92 | 372.58 ± 56.92 | -- | 19.06 ± .67 | 22.22 ± 0.92 |
| 19G294 Group | 228.60 ± 74.30** | 127.18 ± 47.60** | 66% | 19.28 ± 0.60 | 23.00 ± 1.19 |

*: P < 0.05, vs. Model Group.
a: indicating that TGI was calculated based on
tumor weight on Day 57 after cell inoculation.

### Example 20. Efficacy of Humanized Bispecific Antibody 19G294 in NOD/SCID Mice Bearing A498 (renal cancer) Xenografts

Methods: NOD/SCID mice weighing 18-22 g and aged approximately 5-7 weeks were selected. A suspension of A498 (1×107 cells/0.1 mL) was thoroughly mixed with a suspension of PBMCs (1×107 cells/0.1 mL) at a 1:1 volume ratio, and 0.2 mL of the mixture was inoculated subcutaneously into each mouse. The animals were randomly divided into two groups based on body weight: Model Group (PBS), 19G294 Group (30 μg/mouse/dose), with five animals in each group.

The administration route and dosing schedule were the same as in Example 17.

General clinical observations, body weight, tumor volume, and tumor weight measurements were conducted as in Example 17.

Conclusions: At a dose of 30 μg/mouse, 19G294 completely inhibited tumor growth in mice bearing human renal cancer xenografts. The results are shown in FIG. 10 and Table 21. At the dose of 30 μg/mouse, no body weight loss was observed in the animals, and the treatment was well tolerated during the study.

TABLE 21
Tumor growth and body weight in mice bearing subcutaneous A498 human renal cancer xenografts

| | D61 | D61 | | D1 | D61 |
|---|---|---|---|---|---|
| | tumor volume | Tumor weight | TGI a | body weight | body weight |
| Group | (mm3) | (mg) | (%) | (g) | (g) |
| Model Group | 1648.94 ± 444.85 | 1105.04 ± 342.68 | -- | 20.68 ± 0.81 | 24.06 ± 0.81 |
| 19G294 Group | 0.00 ± 0.00** | 0.00 ± 0.00** | 100% | 20.50 ± 0.50 | 24.18 ± 0.93 |

*: P < 0.05, vs. Model Group;
**: P < 0.01, vs. Model Group.
a: indicating that TGI was calculated based on
tumor weight on Day 61 after cell inoculation.

### Example 21. Efficacy of Humanized Bispecific Antibodies 18G272, 19G294, and Others in NOD/SCID Mice Bearing OS-RC-2 (Renal Cancer) Xenografts

### Experiment 1

Methods: NOD/SCID mice weighing 18-22 g and aged approximately 5-7 weeks were selected. A suspension of OS-RC-2 (5×106 cells/0.1 mL) was thoroughly mixed with a suspension of PBMCs (1×107 cells/0.1 mL) at a 1:1 volume ratio, and 0.2 mL of the mixture was inoculated subcutaneously into each mouse. The animals were randomly divided into three groups based on body weight: Model Group (PBS), 17G29 Group (15 μg/mouse/dose), 19G295 Group (15 μg/mouse/dose), with eight animals in each group.

The administration route and dosing schedule were the same as in Example 17.

General clinical observations, body weight, and tumor volume measurements were conducted as in Example 17.

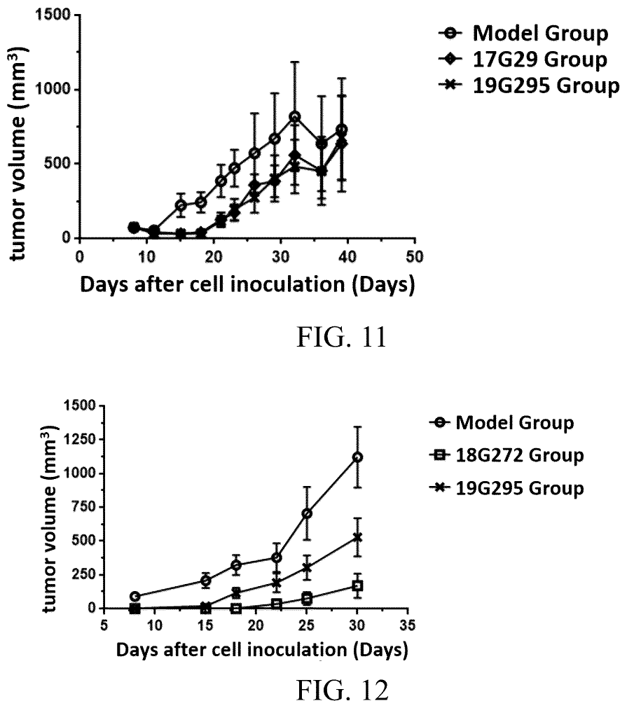

Conclusions: At a dose of 15 μg/mouse, both 17G29 and 19G295 significantly inhibited tumor growth on Day 15, as shown in FIG. 11; however, by Day 39, neither 17G29 nor 19G295 was able to inhibit tumor growth, with no statistically significant difference between the two groups. In addition, the OS-RC-2 human renal cancer model caused animal deaths during the study period, whereas treatment with 17G29 or 19G295 at 15 μg/mouse prolonged the median survival of mice, with no statistically significant difference between the two groups.

TABLE 22
Tumor growth and body weight in mice bearing subcutaneous OS-RC-2 human renal cancer xenografts

| | D15 | D39 | D1 | D15 | D39 |
|---|---|---|---|---|---|
| | tumor volume | tumor volume | body weight | body weight | survival |
| Group | (mm3) | (mm3) | (g) | (g) | rate |
| Model Group | 222.35 ± 79.19 | 731.61 ± 296.48 | 18.92 ± 0.50 | 19.24 ± 0.18 | 3/8 |
| 17G29 Group | 30.48 ± 15.81* | 633.78 ± 248.84 | 18.91 ± 0.51 | 19.74 ± 0.40 | 3/8 |
| 19G295 Group | 31.77 ± 13.63* | 676.71 ± 261.55 | 19.15 ± 0.35 | 20.05 ± 0.43 | 6/8 |

*: P < 0.05, vs. Model Group;
**: P < 0.01, vs. Model Group.
After Day 15, animals in each group began to
die or were euthanized due to tumor growth.

### Experiment 2

Methods: NOD/SCID mice weighing 18-22 g and aged approximately 5-7 weeks were selected. A suspension of OS-RC-2 (3×106 cells/0.1 mL) was thoroughly mixed with a suspension of PBMCs (6×106 cells/0.1 mL) at a 1:1 volume ratio, and 0.2 mL of the mixture was inoculated subcutaneously into each mouse. The animals were randomly divided into three groups based on body weight: Model Group (PBS), 18G272 Group (30 μg/mouse/dose), 19G295 Group (30 μg/mouse/dose), with six animals in each group.

The administration route and dosing schedule were the same as in Example 17.

General clinical observations, body weight, and tumor volume measurements were conducted as in Example 17.

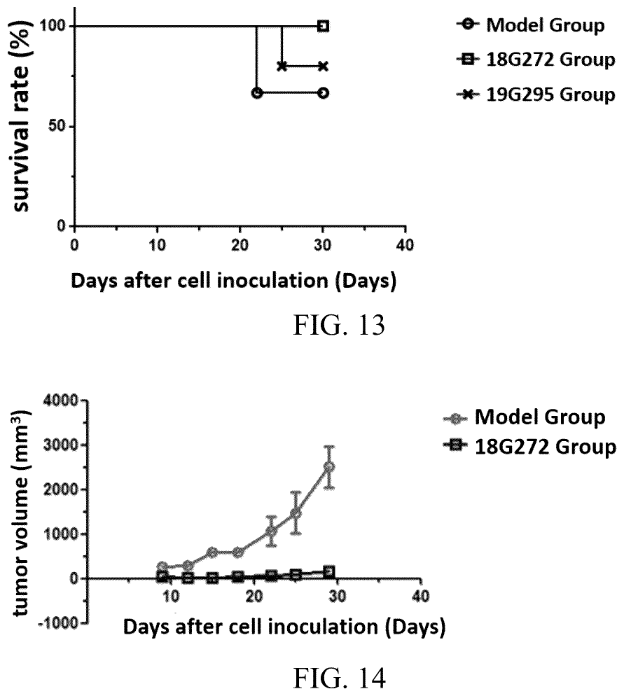

Conclusions: Compared with the Model Group, 18G272 at a dose of 30 μg/mouse significantly inhibited tumor growth on Day 22, while 19G295 showed a trend of tumor inhibition but without a significant difference. By Day 30, 18G272 Group still exhibited strong tumor inhibition, with a highly significant difference, whereas 19G295 Group showed a trend of tumor inhibition but no significant difference compared with Model Group (see FIG. 12 and Table 23). The OS-RC-2 human renal cancer model caused animal deaths during the study period; however, no deaths occurred in 18G272 Group at 30 μg/mouse/dose, whereas deaths were observed in 19G295 Group (see FIG. 13 and Table 23). In summary, the antitumor effect of 18G272 was superior to that of 19G295.

TABLE 23
Tumor growth and survival rate in mice bearing subcutaneous OS-RC-2 human renal cancer xenografts

| | D22 | D30 | | D1 | D22 | D31 |
|---|---|---|---|---|---|---|
| | tumor volume | tumor volume | TCIa | body weight | body weight | survival |
| Group | (mm3) | (mm3) | (%) | (g) | (g) | rate |
| Model Group | 376.36 ± 107.37 | 1121.61 ± 225.44 | | 17.37 ± 0.92 | 12.93 ± 1.53 | 2/3 |
| 18G272 Group | 32.59 ± 32.59** | 168.39 ± 90.70** | 91.3% | 18.12 ± 0.38 | 20.40 ± 0.45 | 5/5 |
| 19G295 Group | 190.86 ± 69.80 | 528.05 ± 140.97 | 49.3% | 18.06 ± 0.34 | 18.22 ± 1.11 | 4/5 |

*: P < 0.05, vs. Model Group;
**: P < 0.01, vs. Model Group.

After Day 22, animals in each group began to die or were euthanized due to tumor growth.
a: indicating that TGI was calculated based on tumor volume on Day 22 after cell inoculation.

### Experiment 3

Methods: NOD/SCID mice weighing 18-22 g and aged approximately 5-7 weeks were selected. A suspension of OS-RC-2 (3×106 cells/0.1 mL) was thoroughly mixed with a suspension of PBMCs (6×106 cells/0.1 mL) at a 1:1 volume ratio, and 0.2 mL of the mixture was inoculated subcutaneously into each mouse. The animals were randomly divided into two groups based on body weight: Model Group (PBS), 18G272 Group (30 μg/mouse/dose), with six animals in each group.

The administration route and dosing schedule were the same as in Example 17.

General clinical observations, body weight, tumor volume, and tumor weight measurements were conducted as in Example 17.

Conclusions: At a dose of 30 μg/mouse, 18G272 significantly inhibited tumor growth in mice bearing OS-RC-2 human renal cancer xenografts, as shown in FIG. 14 and Table 24. The OS-RC-2 human renal cancer model caused animal deaths during the study period; under the conditions of this study, treatment with 186272 at 30 μg/mouse significantly prolonged the median survival time (MST) of the mice, reduced the mortality rate, and was well tolerated during the treatment period (see Table 24).

TABLE 24
Tumor growth, body weight and survival rate in mice bearing subcutaneous OS-RC-2 human renal cancer xenografts

| | D15 | D29 | | DI | D15 | D31 |
|---|---|---|---|---|---|---|
| | tumor volume | tumor weight | TCIa | body weight | body weight | survival |
| Group | (mm3) | (mg) | (%) | (g) | (g) | rate |
| Model Group | 598.55 ± 80.50 | 1656.15 ± 147.85 | -- | 18.33 ± 0.18 | 18.07 ± 0.94 | 2/6 |
| 18G272 Group | 12.87 ± 12.87** | 96.80 ± 38.41 | 94.2% | 18.30 ± 0.19 | 19.43 ± 0.67 | 6/6 |

*: P < 0.05, vs. Model Group;
**: P < 0.01, vs. Model Group.

After Day 15, animals in each group began to die or were euthanized due to tumor growth.
a: indicating that TGI was calculated based on tumor weight on Day 29 after cell inoculation.

### Experiment 4

Methods: NOD/SCID mice weighing 18-22 g and aged approximately 5-7 weeks were selected. A suspension of OS-RC-2 (3×106 cells/0.1 mL) was thoroughly mixed with a suspension of PBMCs (6×106 cells/0.1 mL) at a 1:1 volume ratio, and 0.2 mL of the mixture was inoculated subcutaneously into each mouse. The animals were randomly divided into two groups based on body weight: Model Group (PBS), 19G294 Group (30 μg/mouse/dose), with five animals in each group.

The administration route and dosing schedule were the same as in Example 17.

General clinical observations, body weight, and tumor volume measurements were conducted as in Example 17.

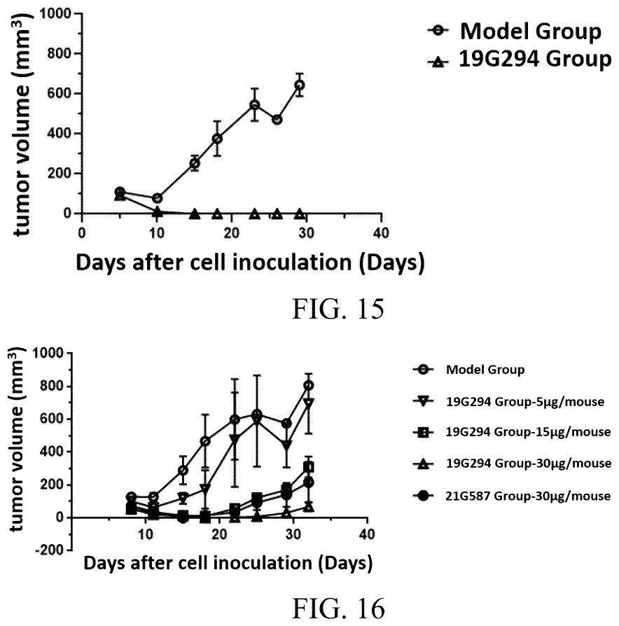

Conclusions: At a dose of 30 μg/mouse, 19G294 significantly inhibited tumor growth in mice bearing OS-RC-2 human renal cancer xenografts, as shown in FIG. 15 and Table 25. The OS-RC-2 human renal cancer model caused animal deaths during the study period; under the conditions of this study, treatment with 19G294 reduced the mortality rate, prolonged the survival time of the mice, and was well tolerated during the treatment period (see Table 25).

TABLE 25
Tumor growth, body weight and survival rate in mice bearing subcutaneous
OS-RC-2 human renal cancer xenografts

| | D23 | D29 | D1 | D23 | D29 |
|---|---|---|---|---|---|
| | tumor volume | tumor volume | body weight | body weight | survival |
| Group | (mm3) | (mm3) | (g) | (g) | rate |
| Model Group | 545.91 ± 80.93 | 645.40 ± 56.68 | 19.04 ± 0.85 | 16.22 ± 1.49 | 2/5 |
| 19G294 Group | 0.00 ± 0.00** | 0.00 ± 0.00** | 18.76 ± 0.57 | 20.34 ± 0.52* | 5/5 |

*: P < 0.05, vs. Model Group;
**: P < 0.01, vs. Model Group.
After Day 23, animals in each group began to die or were euthanized.

Based on the results of the four aforementioned experiments, in the OS-RC-2 (renal cancer) xenograft model in NOD/SCID mice, in terms of efficacy, 17G29 was comparable to 19G295; 19G295 was inferior to 18G272; 19G294 was comparable to 18G272, and both able to significantly inhibit or even completely inhibit tumor growth; in terms of safety, 19G295 was comparable to 17G29; 19G295 was inferior to 18G272; 19G294 was comparable to 18G272. These results indicate that 18G272 and 19G294 have superior in vivo efficacy and safety compared with 17G29.

### Example 22. Efficacy of Different Doses of Humanized Bispecific Antibodies 19G294 and 21G587 in NOD/SCID Mice Bearing OS-RC-2 (Renal Cancer) Xenografts

Methods: NOD/SCID mice weighing 18-22 g and aged approximately 5-7 weeks were selected. A suspension of OS-RC-2 (3×106 cells/0.1 mL) was thoroughly mixed with a suspension of PBMCs (6×106 cells/0.1 mL) at a 1:1 volume ratio, and 0.2 mL of the mixture was inoculated subcutaneously into each mouse. The animals were randomly divided into two groups based on body weight: Model Group (PBS), 19G294 Group (5 μg/mouse/dose), 19G294 Group (15 μg/mouse/dose), 19G294 Group (30 μg/mouse/dose), 21G587 Group (30 μg/mouse/dose), with three animals in each group.

The administration route and dosing schedule were the same as in Example 17.

General clinical observations, body weight, tumor volume, and tumor weight measurements were conducted as in Example 17.

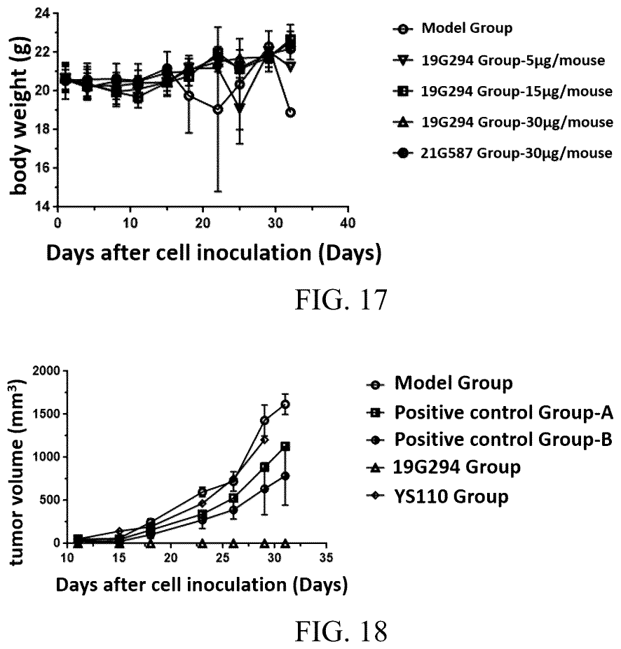

Conclusions: 19G294 at doses of 15 and 30 μg/mouse, and 21G587 at 30 μg/mouse, significantly inhibited tumor growth in the OS-RC-2 human renal cancer xenograft model. The antitumor efficacy of 19G294 exhibited a dose-dependent trend across the low, medium, and high dose groups. The OS-RC-2 human renal cancer model caused animal deaths during the study period; under the conditions of this study, 19G294 (5, 15, and 30 μg/mouse) and 21G587 (30 μg/mouse) improved the decline in animal condition associated with tumor growth, reduced mortality rate, and were well tolerated during the treatment period. The results are shown in FIGS. 16, 17, and Table 26.

TABLE 26
Tumor growth, body weight and survival rate in mice bearing subcutaneous
OS-RC-2 human renal cancer xenografts

| | D18 | D33 | | D1 | D18 | D33 |
|---|---|---|---|---|---|---|
| | tumor volume | tumor weight | TGIa | body weight | body weight | survival |
| Group | (mm3) | (mg) | (%) | (g) | (g) | rate |
| Model Group | 467.29 ± 160.94 | 471.60 ± 0.00 | - | 20.53 ± 0.93 | 19.77 ± 1.92 | 1/3 |
| 19G294 Group | 173.34 ± 116.15 | 311.90 ± 44.42 | 34 | 20.67 ± 0.72 | 21.20 ± 0.64 | 2/3 |
| -5 μg/mouse | | | | | | |
| 19G294 Group | 10.21 ± 10.21 * | 138.87 ± 38.01 | 71 | 20.67 ± 0.67 | 20.77 ± 0.48 | 3/3 |
| -15 μg/mouse | | | | | | |
| 19G294 Group | 0.00 ± 0.00 * | 34.20 ± 21.55 | 93 | 20.67 ± 0.61 | 21.10 ± 0.57 | 3/3 |
| -30 μg/mouse | | | | | | |
| 21G587 Group | 15.84 ± 15.84 * | 122.40 ± 61.17 | 74 | 20.60 ± 0.51 | 21.03 ± 0.43 | 3/3 |
| -30 μg/mouse | | | | | | |

*: P < 0.05, vs. Model Group;
**: P < 0.01, vs. Model Group.
After Day 18, animals began to die or were euthanized.
a: indicating that TGI was calculated based on
tumor weight on Day 33 after cell inoculation.

### Example 23. Efficacy of Humanized Bispecific Antibody 19G294, Axitinib Combined with BAVENCIO, YS110 in NOD/SCID Mice Bearing OS-RC-2 (Renal Cancer) Xenografts

Methods: NOD/SCID mice weighing 18-22 g and aged approximately 5-7 weeks were selected. A suspension of OS-RC-2 (3×106 cells/0.1 mL) was thoroughly mixed with a suspension of PBMCs (6×106 cells/0.1 mL) at a 1:1 volume ratio, and 0.2 mL of the mixture was inoculated subcutaneously into each mouse. The animals were randomly divided into five groups based on body weight: Model Group (PBS), Positive Group-A (BAVENCIO, 1.8 mg/mouse/dose; Axitinib, 30 μg/mosue/dose), Positive Group-B (BAVENCIO, 1.8 mg/mosue/dose; Axitinib, 60 μg/mosue/dose), 19G294 Group (30 μg/mouse/dose), YS110 Group (205 μg/mouse/dose), with three-four animals in each group. Axitinib was administered by oral gavage, whereas BAVENCIO, the test article, and PBS were administered via intravenous injection. Administration of axitinib, 19G294, and PBS started on the day of inoculation, with dosing for five consecutive days, and after a two-day treatment-free interval, a second course of dosing was conducted (dosing for five consecutive days), the dosing frequency was once daily (a total of 10 doses). BAVENCIO administration started on the day of inoculation, the dosing frequency was once per week, with dosing for two consecutive weeks (a total of 2 doses). YS110 administration started on the day of inoculation, the dosing frequency was twice per week, with dosing for two consecutive weeks (a total of 4 doses).

General clinical observations, body weight, tumor volume, and tumor weight measurements were conducted as in Example 17.

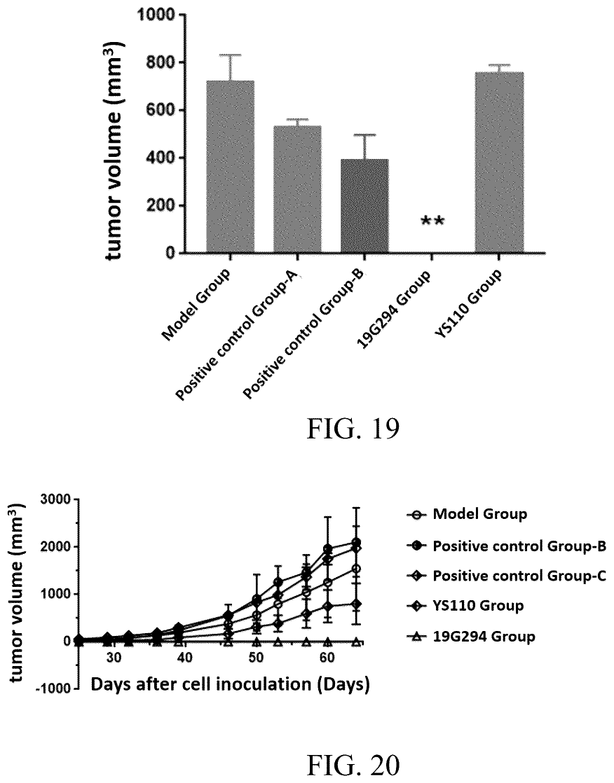

Conclusions: 19G294 at a dose of 30 μg/mouse significantly inhibited tumor growth in the OS-RC-2 human renal cancer xenograft model. BAVENCIO (1.8 mg/mouse) in combination with axitinib (30 μg/mouse or 60 μg/mouse) or YS110 (205 μg/mouse) did not significantly inhibit tumor growth in the OS-RC-2 model. The OS-RC-2 human renal cancer model caused marked body weight loss or deaths in animals during the study period. Under the present experimental conditions, 196294 (30 μg/mouse) improved the decline in animal condition associated with tumor growth, reduced mortality rate, and was well tolerated during the treatment period. In contrast, BAVENCIO in combination with axitinib (both dose groups) or YS110 showed no such improvement. In summary, under the present experimental conditions, the efficacy of 19G294 was superior to that of BAVENCIO combined with axitinib or YS110. The results are shown in FIGS. 18, 19 and Table 27.

TABLE 27
Tumor growth, body weight and survival rate in mice bearing subcutaneous
OS-RC-2 human renal cancer xenografts

| | D26 | D31 | | DI | D26 | D31 |
|---|---|---|---|---|---|---|
| | tumor volume | tumor weight | TGIa | body weight | body weight | survival |
| Groups | (mm3) | (mg) | (%) | (g) | (g) | rate |
| Model Group | 720.68 ± 112.77 | 817.80 ± 35.33 | - | 21.63 ± 0.84 | 16.27 ± 1.28 | 3/3 |
| Positive Group-A | 528.14 ± 5.33 | 656.50 ± 0.00 | 20 | 21.50 ± 0.54 | 17.45 ± 1.01 | 1/4 |
| Positive Group-B | 390.03 ± 108.54 | 435.30 ± 175.60 | 47 | 21.60 ± 0.40 | 19.05 ± 1.82 | 3/4 |
| 19G294 group | 0.00 ± 0.00** | 0.00 ± 0.00* | 100 | 21.73 ± 0.38 | 22.20 ± 0.35 | 3/3 |
| YS110 group | 752.63 ± 39.56 | - | -- | 21.67 ± 0.26 | 16.43 ± 0.55 | 0/3 |

*: P < 0.05, vs. Model Group;
**: P < 0.01, vs. Model Group.
After Day 26, animals began to die or were euthanized.
a: indicating that TGI was calculated based on
tumor weight on Day 31 after cell inoculation.

### Example 24. Efficacy of Humanized Bispecific Antibody 19G294, Axitinib Combined with BAVENCIO, YS110 in NOD/SCID Mice Bearing A498 (Renal Cancer) Xenografts

Methods: NOD/SCID mice weighing 18-22 g and aged approximately 5-7 weeks were selected. A suspension of A498 (7×106 cells/0.1 mL) was thoroughly mixed with a suspension of PBMCs (7×106 cells/0.1 mL) at a 1:1 volume ratio, and 0.2 mL of the mixture was inoculated subcutaneously into each mouse. The animals were randomly divided into five groups based on body weight: Model Group (PBS), Positive Group-B (BAVENCIO, 1.8 mg/mouse/dose; Axitinib, 60 μg/mosue/dose), Positive Group-C (BAVENCIO, 1.8 mg/mosue/dose; Axitinib, 120 μg/mosue/dose), 196294 Group (30 μg/mouse/dose), YS110 Group (205 μg/mouse/dose), with three animals in each group. Axitinib was administered by oral gavage, whereas BAVENCIO, the test article, and PBS were administered via intravenous injection. Administration of axitinib, 19G294, and PBS started on the day of inoculation, with dosing for five consecutive days, and after a two-day treatment-free interval, a second course of dosing was conducted (dosing for five consecutive days), the dosing frequency was once daily (a total of 10 doses). BAVENCIO administration started on the day of inoculation, the dosing frequency was once every two weeks, with dosing for two consecutive weeks (a total of 1 dose). YS110 administration started on the day of inoculation, the dosing frequency was twice per week, with dosing for two consecutive weeks (a total of 4 doses).

General clinical observations, body weight, tumor volume, and tumor weight measurements were conducted as in Example 17.

Organ weight and organ index: After the final measurement, animals were euthanized, and the mouse organs were dissected, rinsed with physiological saline, blotted dry with filter paper, weighed, and photographed. The organ index was calculated as:

organ index=Organ weight/mouse weight×100%
where both organ weight and body weight were measured in grams.

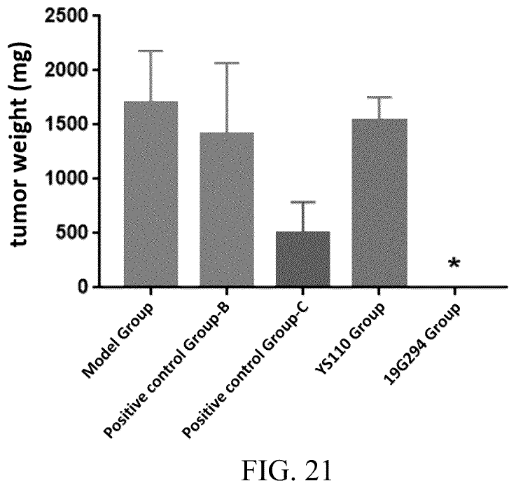

Conclusions: 19G294 at a dose of 30 μg/mouse significantly inhibited tumor growth in the A498 human renal cancer xenograft model. BAVENCIO (1.8 mg/mouse) in combination with axitinib (60 μg/mouse or 120 μg/mouse) or YS110 (205 μg/mouse) did not significantly inhibit tumor growth in the A498 model. 196294 (30 μg/mouse), BAVENCIO in combination with axitinib (both dose groups), and YS110 were well tolerated during the treatment period. 19G294 (30 μg/mouse) and YS110 had no significant effects on the kidney, spleen, liver, lung, or the corresponding organ indices. In contrast, BAVENCIO in combination with axitinib (both dose groups) exerted significant effects on the liver and/or liver index, and showed no significant effects on the kidney, spleen, lung, or the corresponding organ indices. In summary, under the present experimental conditions, the efficacy of 19G294 was superior to that of BAVENCIO in combination with axitinib or YS110, and its safety profile was superior to that of BAVENCIO in combination with axitinib. The results are shown in FIGS. 20, 21 and Tables 28, 29.

TABLE 28
Tumor growth, body weight and survival rate in mice bearing subcutaneous A498 human renal cancer xenografts

| | D64 | D65 | | D1 | D64 |
|---|---|---|---|---|---|
| | tumor volume | tumor weight | TGIa | body weight | body weight |
| Group | (mm3) | (mg) | (%) | (g) | (g) |
| Model Group | 2,324.41 ± 776.85 | 1,699.40 ± 483.70 | - | 20.97 ± 0.84 | 22.47 ± 0.69 |
| Positive Group -B | 2,105.09 ± 728.33 | 1,408.20 ± 661.50 | 17 | 20.90 ± 0.74 | 23.15 ± 1.85 |
| Positive Group -C | 806.49 ± 434.55 | 499.63 ± 288.76 | 71 | 20.90 ± 0.57 | 24.93 ± 0.47 |
| YS110 Group | 1,979.16 ± =97.24 | 1,535.17 ± 219.51 | 10 | 20.93 ± 0.38 | 23.40 ± 1.35 |
| 19G294 Group | 0.00 ± 0.00* | 0.00 ± 0.00* | 100 | 20.93 ± 0.22 | 22.77 ± 0.41 |

*: P < 0.05, vs. Model Group;
**: P < 0.01, vs. Model Group.
a: indicating that TGI was calculated based
on tumor weight on Day 65 after cell inoculation.

TABLE 29-1
Organ weight and organ index statistics in mice bearing subcutaneous A498 human renal cancer xenografts

| Group | Kidney (mg) | Kidney index (g/g) | Spleen (mg) | Spleen index (g/g) |
|---|---|---|---|---|
| Model Group | 240.43 ± 26.28 | 1.070 ± 1.02 | 54.87 ± 5.21 | 0.244 ± 0.15 |
| Positive Group -B | 259.35 ± 24.55 | 1.119 ± 0.17 | 54.35 ± 8.85 | 0.233 ± 0.20 |
| Positive Group -C | 282.40 ± 13.79 | 1.131 ± 0.37 | 63.50 ± 5.13 | 0.254 ± 0.17 |
| YS110 Group | 254.70 ± 13.30 | 1.089 ± 0.07 | 60.10 ± 8.91 | 0.255 ± 0.27 |
| 19G294 Group | 284.83 ± 13.66 | 1.250 ± 0.38 | 63.90 ± 3.11 | 0.280 ± 0.10 |

TABLE 29-2
Organ weight and organ index statistics in mice bearing subcutaneous A498 human renal cancer xenografts

| Group | Liver (mg) | Liver index (g/g) | Lung (mg) | Lung index (g/g) |
|---|---|---|---|---|
| Model Group | 1,070.97 ± 23.17 | 4.772 ± 1.12 | 167.80 ± 6.52 | 0.750 ± 0.59 |
| Positive Group -B | 1,181.60 ± 31.10* | 5.126 ± 2.75 | 166.45 ± 5.05 | 0.722 ± 0.36 |
| Positive Group -C | 1,284.80 ± 34.57** | 5.151 ± 0.45* | 164.83 ± 2.26 | 0.661 ± 0.06 |
| YS110 Group | 1,195.97 ± 131.82 | 5.078 ± 2.89 | 177.57 ± 7.62 | 0.761 ± 0.24 |
| 19G294 Group | 1,177.80 ± 52.22 | 5.169 ± 1.51 | 149.23 ± 5.88 | 0.655 ± 0.16 |

*: P < 0.05, vs. Model Group;
**: P < 0.01, vs. Model Group.

### Example 25. Activity Evaluation of Bispecific Antibody

In the 786-0 cell assay system, 786-0 cells were labeled with a green fluorescent signal using Calcein-AM fluorescent dye. The 786-0 cells were seeded into a U-bottom 96-well cell culture plate at a concentration of 6×10⁵ cells/mL (50 μL per well). Humanized bispecific antibody was added to the corresponding reaction wells at 50 μL per well to reach final concentrations of 100 ng/mL, 10 ng/ml, 1 ng/ml, 0.1 ng/mL, 0.01 ng/mL, and 0.001 ng/mL. Subsequently, PBMCs at a concentration of 9×10⁶ cells/mL were added at an E:T ratio of 15:1 (50 μL per well). The reaction system was incubated at 37 °C under a CO₂ atmosphere for 5 hours. After incubation, the plate was centrifuged and the supernatants were collected and transferred to a new 96-well plate. After a second centrifugation, 80 μL of supernatant from each well was collected and transferred to a black 96-well plate, and fluorescence intensity was measured using a microplate reader at 470 nm excitation wavelength and 515 nm emission wavelength.

The formula for calculating the cell lysis rate was as follows:

Cell lysis rate (%)=[(Vsample−Vvehicle control)/(VTriton-100−Vvehicle control)]×100%

(Vsample is the fluorescence signal reading of the drug-treated group. Vvehicle control is the mean value of the fluorescence signal reading of the blank control group. VTriton-100 is the mean value of the fluorescence signal reading of the positive control group.)

The IC50 values of PBMC cytotoxicity against target cells mediated by antibody samples were calculated using GraphPad Prism 7.00 software based on the cells lysis rate and sample concentrations.

TABLE 30

| IC50 values of humanized bispecific antibody | |
|---|---|
| (derived from mouse antibody No. 79)-mediated | |
| PBMC cytotoxicity against different target cells (pg/ml) | |
| Cell name | 19G300 |
| 786-0 | 6096 |
| OS-RC-2 | 2086 |
| NCI-H226 | 9032 |
| PC-3 | 662.6 |

These results indicate that 19G300 possesses in vitro activity against various tumor cell types.

### Example 26. Binding Ability of Full-Length Humanized Antibody in Mixed Cell System

The binding ability of full-length humanized antibodies 21G428-429 and 21G430-431, derived from mouse antibodies No. 62 and No. 79 in Example 6, was evaluated in a mixed cell system.

786-0 cells were collected, counted, and adjusted to a density of 6×10⁵ cells/mL. PBMCs were collected, counted, and adjusted to a density of 6×10⁵ cells/mL. 50 μL of 786-0 cells and 50 μL of PBMCs were added into an EP tube as a mixed cell system. The antibody was added and diluted to a final concentration of 0.3 μg/mL, mixed well with the mixed cell system, and incubated at 4 °C for 1 hour.

After incubation, the cells were collected by centrifugation, washed twice with PBS, and resuspended in PBS. Goat anti-human IgG fluorescent secondary antibody was added, and the cells were incubated at 4 °C for 1 hour. After incubation, the cells were washed twice with PBS, resuspended in PBS, and analyzed using a BD ACCURI C6 flow cytometer.

The analysis results (FIG. 27) showed that full-length humanized antibodies 21G428-429 and 21G430-431 exhibited significantly higher binding to 786-0 tumor cells than to PBMCs, indicating that these antibodies recognize the CD26 target on tumor cells with greater selectivity than on PBMCs. They preferentially bind to CD26 on tumor cells while showing minimal binding to CD26 on PBMCs, thereby avoiding cytotoxicity to normal cells. Antibodies interact with different epitopes on the antigen via their variable regions to exert specific functions. Since CD26 is expressed on both tumor cells and PBMCs, the substantial difference in binding of 21G428-429 and 21G430-431 to CD26 on tumor cells versus PBMCs is highly significant for enabling the antibodies to exert antitumor effects safely.

### Example 27. Competitive Binding of Antibodies to CD26 Protein

(1) Competitive binding of 19G294 (parent antibody of the anti-CD26 segment: mouse antibody No. 62) and 19G300 (parent antibody of the anti-CD26 segment: mouse antibody No. 79) to CD26 protein

CD26 protein was biotinylated using EZ-link NHS-PEG12-Biotin.

Two SA biosensors were used to immobilize biotinylated CD26, which were designated as Sensor F4 and Sensor G4 (A).

The sensors were equilibrated in 1× Kinetics buffer (B).

Sensor F4 was first exposed to 1× Kinetics buffer containing 500 nM 19G294 and allowed to reach equilibrium (C), and was then exposed to 1× Kinetics buffer containing 500 nM 19G300 (this solution contained 500 nM 19G294) (D).

Sensor G4 was first exposed to 1× Kinetics buffer containing 500 nM 19G300 and allowed to reach equilibrium (C), and was then exposed to 1× Kinetics buffer containing 500 nM 19G294 (this solution contained 500 nM 19G300) (D).

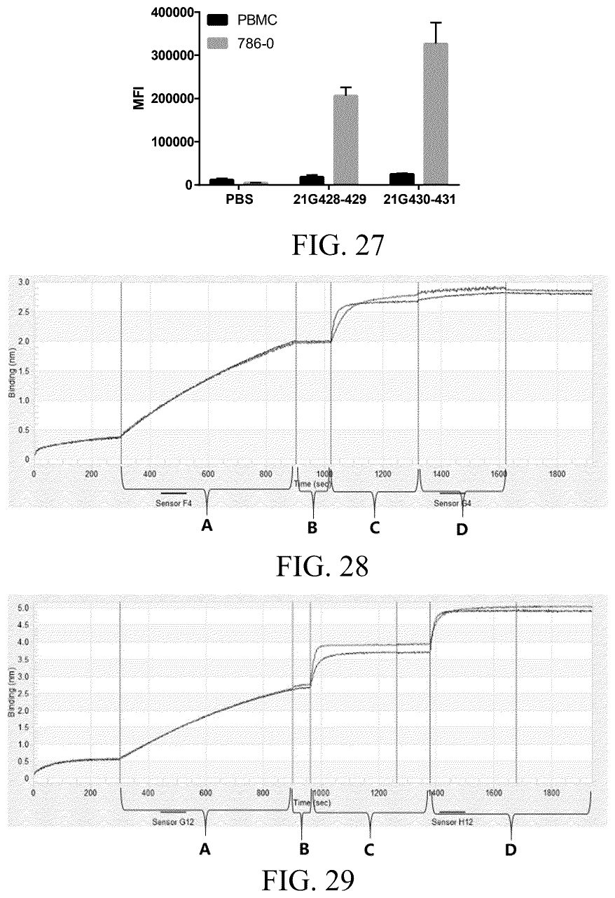

As shown in FIG. 28, when CD26 was first bound by 196294, subsequent binding by 19G300 was substantially inhibited (no clear binding signal in step D). Similarly, when CD26 was first bound by 19G300, subsequent binding by 19G294 was also substantially inhibited (no clear binding signal in step D). These results indicate that 19G294 and 19G300 compete for binding to CD26, suggesting that their epitopes on CD26 partially overlap.
(2) Competitive binding of 19G294 and 17G29 (parent antibody of the anti-CD26 segment: YS110) to CD26 protein

CD26 protein was biotinylated using EZ-link NHS-PEG12-Biotin.

Two SA biosensors were used to immobilize biotinylated CD26, which were designated as Sensor G12 and Sensor H12 (A).

The sensors were equilibrated in 1× Kinetics buffer (B).

Sensor G12 was first exposed to 1× Kinetics buffer containing 500 nM 17G29 and allowed to reach equilibrium (C), and was then exposed to 1× Kinetics buffer containing 500 nM 19G294 (D).

Sensor H12 was first exposed to 1× Kinetics buffer containing 500 nM 19G294 and allowed to reach equilibrium (C), and was then exposed to 1× Kinetics buffer containing 500 nM 17G29 (D).

As shown in FIG. 29, when CD26 was first bound by 19G294, it could still bind to 17G29 subsequently (a clear binding signal in step D). Similarly, when CD26 was first bound by 17G29, it could still bind to 19G294 subsequently (a clear binding signal in step D). These results indicate that 19G294 and 17G29 (whose anti-CD26 segment sequence is identical to YS110 variable region sequence) do not compete for binding to CD26.

### Example 28. Identification of Antibody-Binding Antigen Fragment (1)

### Step 1:

The CD26 protein solution was buffer-exchanged into Tris-HCl (100 mM) solution, and added to 2.5 μL Trypsin solution at 25 μg/50 μL. The mixture was incubated at 37 °C for 17 h for enzymatic digestion.

### Step 2:

Mass spectrometry (MS) database establishment: The tryptic digested products of CD26 obtained in Step 1 were analyzed by MS to obtain the data of digested CD26 fragments.

### Step 3:

The antibody 19G294 was biotinylated using EZ-link NHS-PEG12-Biotin and coupled to an SA biosensor. The biosensor was then immersed in the digested CD26 solution to capture antibody fragments capable of binding, and then eluted using 0.4 μL of 0.1% trifluoroacetic acid (this process was repeated 10 times to collect a total of 40 μL of eluted product).

### Step 4:

The eluted product obtained in Step 3 were analyzed by MS, and the resulting data were compared with the database generated in Step 2 to identify the antibody-binding CD26 fragments.

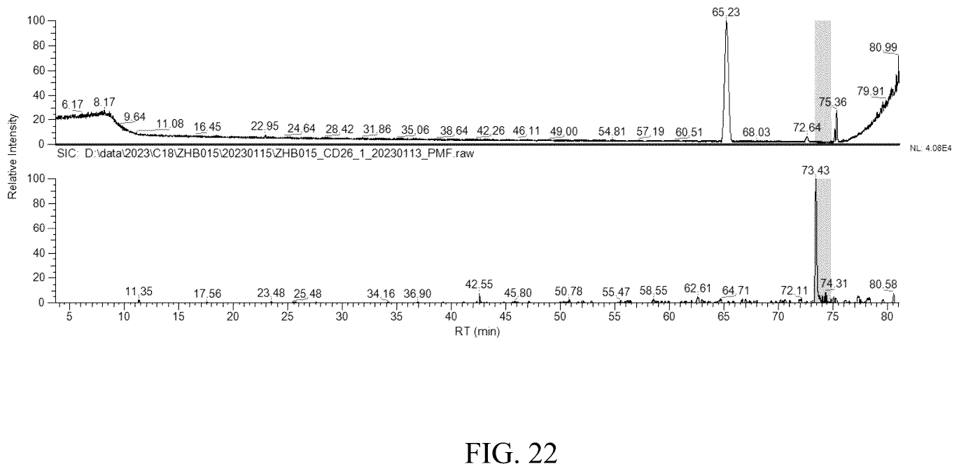

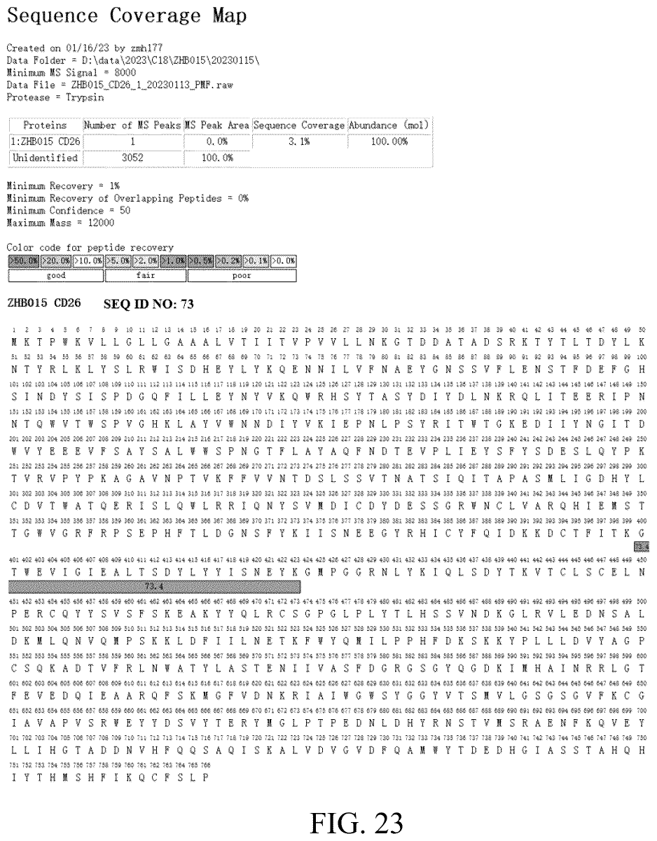

As shown in FIGS. 22, 23 and Table 31, the amino acid sequence coverage analysis indicated that only the peptide GTWEVIGIEALTSDYLYYISNEYK was identified. Therefore, the CD26 fragment bound by 19G294 was determined to be G400-K423 (GTWEVIGIEALTSDYLYYISNEYK, SEQ ID NO: 59). Using the same method described above, the antigen fragment bound by 19G300 was also identified, and was determined to be the same antigen fragment GTWEVIGIEALTSDYLYYISNEYK. In addition, the patent of Y'S Therapeutics (Application No.: CN200680034937.4) discloses information on the antigen fragments bound by anti-CD26 antibody, which is different from the CD 26 region that 19G294 binds to. 19G294 and 19G300 are derived from mouse antibodies No. 62 and No. 79, respectively. The mouse antibodies, the humanized antibodies (21G428-429, 21G430-431) and the bispecific antibodies (such as 19G294 and 19G300) constructed based on the two mouse antibodies have demonstrated superior in vitro and in vivo activity and safety compared with other CD26 antibodies, such as YS110 according to the aforementioned experiments. These experimental results indicate that antibodies binding to the fragment G400-K423 possess advantageous properties.

TABLE 31
Sample binding antigen region peptide fragment information

| | | | | | | | | Avg | | |
|---|---|---|---|---|---|---|---|---|---|---|
| | | | Delta | RT | | Charge | Mono | Mass | Theoretical | MS |
| Peptide Sequence | Modification | Site | (ppm) | (min) | M/Z | State | Mass Exp. | Exp. | Mass | Area |
| GTWEVIGIEALT | None | / | 1.99 | 73.44 | 943.464 | 3 | 2826.3696 | 2827.79 | 2826.364 | 6.58E+03 |
| SDYLYYISNEYK | | | | | | | | | | |

### Example 29: Identification of Antibody-Binding Antigen Fragment of (2)

### 1. Vector Construction

Vectors for displaying the CD26 extracellular domain (29-766aa, SEQ ID NO: 60) or truncated extracellular regions of varying lengths were constructed. The CD26 signal peptide and CD26 transmembrane domain sequence (SEQ ID NO: 63) were fused with the full-length extracellular domain (SEQ ID NO: 60), and then further fused with the F2A sequence (SEQ ID NO: 61) and GFP (SEQ ID NO: 62) sequence. The resulting fusion sequence was codon-optimized for mammalian cells and then gene synthesis was conducted. The gene was amplified by PCR, a BamHI site and a GCCGCCACC (Kozak) sequence were added at the 5′ end of the gene, a stop codon and a SalI site sequence were added at the 3′ end. The PCR products were ligated via the BamHI and SalI restriction sites into the BamHI/SalI double-digested linearized pWPT-GFP vector, replacing the original GFP of the vectors with the CD26 signal peptide-CD26 transmembrane domain-CD26 extracellular domain-F2A peptide-GFP sequence. Upon translation, the CD26 transmembrane domain-CD26 extracellular domain-F2A-GFP was cleaved by the self-cleaving activity of the F2A peptide, retaining GFP intracellularly, while the CD26 extracellular domain was transported and displayed extracellularly.

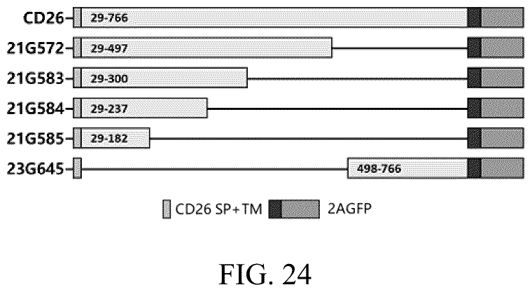

Using a similar construction strategy as that for the full CD26 extracellular domain vectors, the full extracellular domain was replaced with various truncated fragments to construct vectors 21G572 (29-497aa, SEQ ID NO: 64), 21G583 (29-300aa, SEQ ID NO: 66), 21G584 (29-237aa, SEQ ID NO: 67), 21G585 (29-182aa, SEQ ID NO: 68), and 23G645 (498-766aa, SEQ ID NO: 65) (FIG. 24).

The expression vectors were confirmed to be correct by sequencing, and plasmid DNA was prepared using an endotoxin-free plasmid extraction kit.

### 2. Transfection of 293T Cells

The above plasmids were transfected into 293T cells in 6-well plates using the PEI method (PEL: plasmid=10 μg: 2 μg). 293T cells treated with PEI alone were used as the negative control. After 48-72 h, the cells were collected and resuspended in 100 μL at a density of 1×10⁶ cells/mL. Different amounts of 19G294 or 21G428-429 antibodies were used to label the cells at room temperature for 1 h, then the cells were washed three times with 1×PBS and resuspended to 100 μL. Goat anti-human fluorescent secondary antibody was then used to label the cells at room temperature for 1 h, followed by three washes with 1×PBS. The cells were analyzed using a BD Accuri C6 flow cytometer.

### 3. Results and Analysis

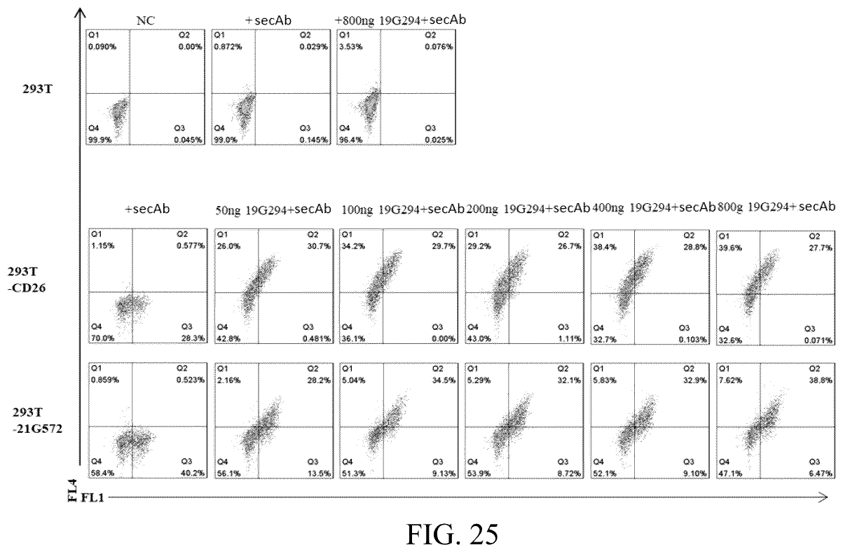

As shown in FIG. 25, 293T cells without vector transfection did not express CD26-F2A-GFP, and no fluorescence signal was detected whether or not the cells were labeled with 19G294 antibody. 293T cells transfected with full-length CD26 extracellular domain (29-766aa)-GFP exhibited approximately 28% GFP-positive cells (FL1 channel positive) even without labeling with 19G294. After labeled with different concentrations of 19G294, the GFP-positive cells also exhibited FL4 channel positivity, indicating that all cells expressing full-length CD26 extracellular domain (29-766 aa)-GFP were bound by 19G294. For 293T cells transfected with 21G572-GFP, approximately 40% of the cells were GFP-positive. After labeling with 19G294, the FL4 channel shift of this cell population consistent with that of full-length CD26 extracellular domain, indicating that the 216572 fragment (CD26 extracellular domain 29-497aa) contains the complete 19G294-binding fragment.

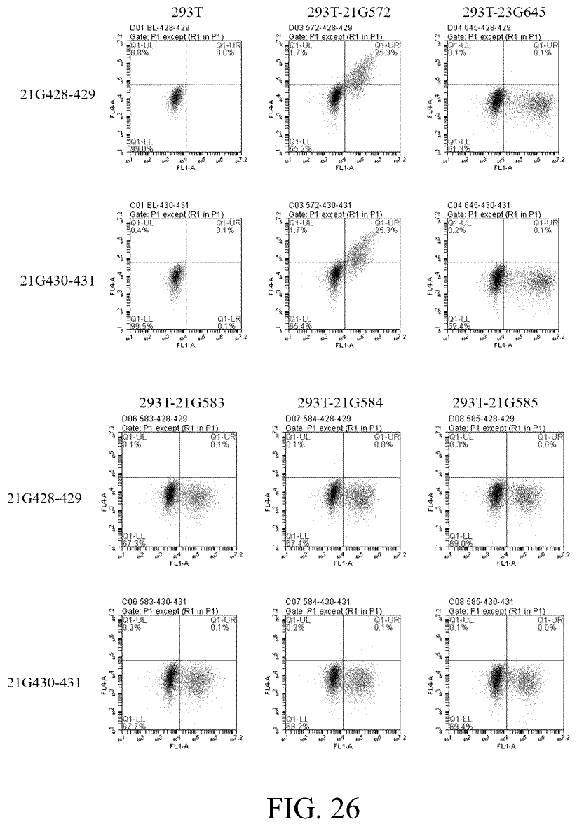

In further studies, 21G572, 21G583, 21G584, 21G585, and 23G645 vectors were individually transfected into 293T cells, which were then labeled with the fully humanized antibody 21G430-431 (derived from mouse antibody No. 79) or the fully humanized antibody 21G428-429 (derived from mouse antibody No. 62), and subsequently labeled with a secondary antibody. The Analysis results showed that 21G430-431 and 21G428-429 exhibited binding to 21G572 consistent with that of 19G294 (FIG. 26), indicating that the binding fragments are the same. In contrast, 21G428-429 did not bind to the CD26 extracellular fragments represented by 21G583 (29-300aa), 21G584 (29-237aa), 21G585 (29-182aa), or 23G645 (498-766aa).

1. An isolated polypeptide, the amino acid sequence of which is identical to or has at least 90% sequence identity to the sequence as set forth in SEQ ID NO: 59 or SEQ ID NO: 64.

2. An antigen-binding protein that binds CD26, wherein the protein recognizes amino acid sequence region of CD26 as set forth in SEQ ID NO: 59 or SEQ ID NO: 64.

3. An antigen-binding protein that binds CD26, wherein the protein competes for binding to CD26 with an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5.

4. The antigen-binding protein according to claim 3, recognizing the same CD26 fragment as that recognized by an antigen-binding protein comprising HCDR1 as set forth in SEQ ID NO: 1, HCDR2 as set forth in SEQ ID NO: 2, HCDR3 as set forth in SEQ ID NO: 3, LCDR1 as set forth in SEQ ID NO: 4, LCDR2 consisting of Arg, Met, and Ser, and LCDR3 as set forth in SEQ ID NO: 5.

5. The antigen-binding protein according to claim 3 or 4, binding to amino acid residues within the amino acid sequence as set forth in SEQ ID NO: 59 or SEQ ID NO: 64.

6. An isolated nucleic acid selected from any one of the following:
a) a DNA encoding an antigen-binding protein, wherein the antigen-binding protein is a CD-26 binding antigen-binding protein according to any one of claims 2 to 5;
b) a DNA encoding a polypeptide, wherein the polypeptide has an amino acid sequence at least 90% sequence identity to SEQ ID NO: 59 or SEQ ID NO: 64;
c) a DNA that is fully complementary to the DNA of a) or b);
d) an RNA of a, b, or c, wherein U substitutes for T.

7. A vector comprising the nucleic acid according to claim 6.

8. A cell comprising the vector according to claim 7.

9. A composition comprising an antigen-binding protein according to any one of claims 2 to 5, a nucleic acid according to claim 6, a vector according to claim 7, or a cell according to claim 8.

10. A method for treating a disease associated with high-level CD26, comprising administering to a subject an effective amount of an antigen-binding protein according to any one of claims 2 to 5, a nucleic acid according to claim 6, a vector according to claim 7, or a cell according to claim 8; wherein the disease associated with high-level CD26 includes, but is not limited to, tumor, and wherein the tumor with high-level CD26 includes, but is not limited to, renal cancer, mesothelioma, lung cancer, liver cancer, and prostate cancer; and wherein the antibody or antigen-binding fragment is administered alone or in combination with other anti-cancer agents.
